(19) United States
(12) Patent Application Publication
(10) Pub. No.: US 2023/0310699 A1
(43) Pub. Date: Oct. 5, 2023

(54) HAEMOSTATIC MATERIAL

(71) Applicant: The Australian National University, Acton (AU)

(72) Inventors: Takuya TSUZUKI, Baldivis (AU); Lucy Anne COUPLAND, Chifley (AU); Elmira MOHAMED, Nicholls (AU)

(21) Appl. No.: 18/009,554

(22) PCT Filed: Jun. 10, 2021
(86) PCT No.: PCT/AU2021/050591

§ 371 Date: Dec. 9, 2022

(30) Foreign Application Priority Data
Jun. 10, 2020 (AU) 2020901904

(51) Int. Cl. A61L 24/08

(52) U.S. Cl. CPC A61L 24/08; A61L 2400/04; A61L 2400/12

(57) ABSTRACT

A haemostatic composition comprising a non-acidic meshed network of fibrous material, wherein the network comprises fibres with a mean diameter (Dso) no greater than 1 μm, an aspect ratio (mean fibre length/mean fibre diameter) of at least 100, and wherein said meshed network has a specific surface area of at least 1O m²/g, and a gel point no greater than 3 g/L.

### CROSS-REFERENCE TO RELATED APPLICATIONS

This application is the National Stage entry of International Application No. PCT/AU2021/050591, filed 10 Jun. 2021, which claims priority to Australian Patent Application No. 2020901904, filed 10 Jun. 2020.

### FIELD OF THE INVENTION

The present invention relates to improved materials for controlling bleeding from external or internal wounds.

### DESCRIPTION OF RELATED ART

Internal and external haemorrhage, typically resulting from traumatic injury but also during childbirth and surgery, as well as associated haemorrhagic shock, are major causes of death. Furthermore, serious trauma can result in bleeding disorder (coagulopathy), which impairs blood clotting. Additionally, an increased risk of bleeding, including thrombocytopenia, occurs with many drugs, diseases and genetic disorders with spontaneous bleeding (such as from the nose or gums) or excessive blood loss following minor injury or surgery.

The normal response to tissue injury involving blood vessels is a process called haemostasis, also known as blood coagulation. This process involves a complex series of reactions involving plasma coagulation factors, platelets and, to a lesser extent, red blood cells. In severe or uncontrolled bleeding, the body's natural coagulation processes are not able to achieve haemostasis due to the extent of blood vessel injury and/or underlying coagulation abnormalities. Numerous haemostatic agents have been developed that target different aspects of the coagulation process and in different application forms, such as topical, intracavitary and intravenous. Commercialized topical and intracavitary haemostatic agents, as described below, are composed of materials such as fibrin-based glues or sealants, zeolite powders, kaolin-impregnated gauze (KG), gelatin foams and oxidized regenerated cellulose (ORC) films. However, there are limitations associated with these materials including acidity causing burns, bystander cell damage and/or inflammation, non-biocompatibility, high cost and short shelf-life. In order to overcome one or more of these drawbacks, many recent attempts have been made to enhance or modify these materials.

One traditional haemostatic material is cellulose-based gauze or balls (such as cotton balls). In order to obtain better haemostasis, the various materials have been developed either as an additive to gauze or as stand-alone products. However, the current haemostatic materials have deficiencies as described below.

Oxidised cellulose: the most widely used haemostatic product in surgery is Surgicel®, which is made of oxidized, regenerated cellulose fibres. It is widely believed that the acidic nature of oxidized cellulose fibres is important in inducing effective blood-clotting. Oxidised celluloses are bio-absorbable/compatible. However, as side effects, they cause haemolysis (red blood cell destruction) as a part of its blood-clot formation mechanism, and induces inflammation and, consequently, delayed wound healing.

Clay and other minerals: the US military uses Combat Gauze® (a kaolin-impregnated gauze) as its main hemostatic agent. These materials enhance clot formation by water-absorption. Kaolin also activates the intrinsic pathway of coagulation (Factor XII). However, these materials do not aid in countering coagulopathy, are not bio-absorbable or compatible, may enter the blood stream and cause thrombotic complications, and need to be removed before surgery. Some of these materials, such as QuickClot ACS+ (zeolite impregnated gauze) generates heat via an exothermic reaction on exposure to moisture with 25% of patients reporting pain following application.

Chitosan: a positively charged polysaccharide (an, at least, partially deacetylated form of chitin) that binds to negatively-charged blood components and forms a physical barrier by adhering to wet issues. It is bio-absorbable, however, the procoagulant activity of the chitosan material Celox™ has been reported to be less effective than oxidised cellulose or clay-based haemostatic products.

Regenerated cellulose: See “oxidised cellulose” for oxidised regenerated cellulose (Surgicel®). Chemically treated regenerated cellulose (ActCel® gauze) expands 3-4 times its original size when in contact with blood, thus sealing off damaged vessels and aiding clotting. It is bio-absorbable and does not cause inflammation. Commercial products include large micron-scale fibres, but large-scale production of nano-scale regenerated cellulose has not been developed.

Collagen: a protein that forms a physical matrix to bind clotting factors and is bio-absorbable. However, it is not effective in the case of thrombocytopenia blood disorder (patients with low platelet counts).

Thrombin: a natural enzyme in the human body which catalyses the conversion of fibrinogen to fibrin and activates procoagulant Factors V, VIII, XI, and XIII in natural haemostasis. It is normally used in a form of solution or impregnated into gauze. However, it is expensive and not suitable for patients with fibrinogenemia (patients who have excess fibrinogen) as intravascular clotting or death can occur in case of thrombin entry into larger calibre vessels.

There is a need for improved tools for the management of blood loss by first aid responders and surgeons which overcome one or more of the disadvantages associated with the existing materials, such as, for example, inflammation, localised burning, cell lysis, and bio-compatibility/absorbability.

### SUMMARY OF THE INVENTION

The present investigations have surprisingly shown that a three-dimensional non-acidic fibrous mesh with a high aspect ratio (mean fibre length/mean fibre diameter), high specific surface area and low gel point provides faster blood clotting than commonly used commercial products. These materials have been found to induce fibrin formation through plasma coagulation, consequently activating platelets trapped within its mesh-like structure, and to be more effective than other common haemostatic materials in the promotion of coagulation of blood from patients with severe thrombocytopenia, without the addition of other reagents.

Thus, according to an aspect of the invention, there is provided a composition for controlling bleeding, said composition comprising a non-acidic meshed network of fibrous material, wherein the network comprises fibres with a mean diameter (D₅₀) no greater than 1 μm, an aspect ratio (mean fibre length/mean fibre diameter) of at least 100, and wherein the meshed network of fibrous material has a specific surface area of at least 10 m²/g, and a gel point no greater than 3 g/L.

In certain embodiments, the fibrous material is derived from a biopolymer. In further embodiments, the biopolymer is a non-oxidised insoluble polysaccharide. In yet further embodiments the polysaccharide comprises a β-linked backbone. In yet further embodiments the polysaccharide is cellulose. In yet further embodiments the cellulose is a high molecular weight cellulose such as α-cellulose.

In certain embodiment, the meshed network of fibrous material is prepared by mechanical processing, such as ball milling of at least partially fractionated biomass rich in high molecular weight cellulose.

According to certain embodiments the meshed network comprises fibres with a mean diameter less than 100 nm, an aspect ratio of at least 120, and has a specific surface area of at least 13 m²/g, and a gel point no greater than 1.5 g/L.

According to certain embodiments the meshed network comprises fibres with a mean diameter less than 50 nm, an aspect ratio of at least 150, and has a specific surface area of at least 15 m²/g, and a gel point no greater than 1.2 g/L.

According to a specific aspect of the invention, there is provided a composition for controlling bleeding, said composition comprising a non-oxidised meshed network of cellulose fibres, said cellulose fibres having a mean diameter (D₅₀) less than 100 nm, optionally less than 50 nm, an aspect ratio (mean fibre length/mean fibre diameter) of at least 120, optionally at least 150, and wherein said meshed network has a specific surface area of at least 13 m²/g, optionally at least 15 m²/g, and a gel point no greater than 1.5 g/L, optionally no greater than 1.2 g/L. According to a specific embodiment, the meshed network may be prepared by ball-milling of at least partially fractionated biomass rich in high molecular weight cellulose having a mean degree of polymerization of at least 1000.

According to another aspect of the present invention, there is provided a method for controlling bleeding from a surface, the method comprising applying to said surface a composition according to the invention. According to a related aspect of the present invention, there is provided the use of a composition according to the invention for the manufacture of a medicament for controlling bleeding.

According to another specific aspect of the present invention, there is provided a composition for controlling bleeding, said material comprising a non-oxidised meshed network of chitin fibres, said chitin fibres having a mean diameter (D₅₀) less than 100 nm, an aspect ratio (mean fibre length/mean fibre diameter) of at least 100, and wherein said meshed network has a specific surface area of at least 13 m²/g, and a gel point no greater than 1.5 g/L. According to an embodiment of this aspect, there is provided a method for controlling bleeding from a surface, said method comprising applying to said surface a composition comprising a non-oxidised meshed network of chitin fibres according to the invention. According to an embodiment of this aspect, there is provided the use of a composition comprising a non-oxidised meshed network of chitin fibres according to the invention for the manufacture of a medicament for controlling bleeding.

### BRIEF DESCRIPTION OF THE DRAWINGS

FIG. 1—Effect of the milling time on fibre morphology. (A) Aspect ratio obtained from sedimentation technique, and BET specific surface area of the cellulose milled for 0 to 180 min. (B) FESEM images of the commercial hemostats (ORC and KG). (C) FESEM images of the cellulose milled for 0 to 180 min, where the sample was dispersed in DI water (0.01 wt. %) first and then one drop of the dispersion was dried on a silicon substrate.

FIG. 2—Effect of milling on the gel point and aspect ratio of fibers. (A) apparent aspect ratio. (B) gel point of ball-milled α-cellulose, as a function of milling time.

FIG. 3—Hemostatic behavior of cellulose nanofibers in comparison with commercial hemostats in suspension and dry forms. (A) Coagulation parameters (CT=clotting time, CFT=clot formation time, α angle and MCF=maximum clot firmness.) obtained from non-activated ROTEM assays (NATEM) in the presence of CellNFs (cellulose nanofibers) with different milling times (eg. CellNF0, CellNF90, CellNF180, with milling time expressed in minutes), expressed as a percentage of the control blood, and in comparison to ORC. 50 μL of CellNFs and ORC suspensions in PBS at 1 wt. % were added to 300 μL blood. 50 μL PBS was added to the same volume of blood as a control. Mean values (n=4-9) are shown with ±SE (standard error) and one-way ANOVA was performed (*p<0.05). (B) As in A, except that different concentrations of CellNF90 suspensions were compared. Mean values (n=3-7) are shown with ±SE (standard error) and one-way ANOVA was performed (*p<0.05). (C) As in A, except that CellNF90 is compared with ORC and KG in a dry form at the same sample-to-blood weight ratio. Untreated blood was used as a control. Mean values (n=3-6) are shown with ±SE (standard error) and one-way ANOVA was performed (*p<0.05). (D) SEM images of the blood clot of control (PBS), CellNF90, ORC and KG.

FIG. 4—Contribution of plasma coagulation in the hemostatic performance of CellNFs. (A) Coagulation parameters (CT=clotting time and A10=clot amplitude at 10 minutes) obtained using the NATEM and non-activated FibTEM assay (NAFibTEM in which platelets are neutralized), relative to control blood in the presence of CellNF0, CellNF90, and CellNF180 and in comparison to ORC. The platelet contribution to the clot was calculated by subtracting the A10 value obtained from the NAFibTEM assay from that of the NATEM assay. Mean values (n=3-7) are shown with ±SE (standard error) and one-way ANOVA was performed (*p<0.05). (B) Comparison of clotting profiles of blood in the presence of CellNF90 with and without addition of calcium chloride. Coagulation parameters are presented as the difference between the 2 samples. (C) Clotting profiles obtained using the NATEM assay and plasma deficient of the coagulation Factors IX, XI or XII in the presence and absence of CellNF90 suspended in PBS at 1 wt. %. (D) Effect on clotting time of a titration of heparin into whole blood with/without addition of CellNF90 (left axis) with difference expressed as % reduction in clotting time (right axis). Mean values (n=3-7) are shown with ±SE (standard error) and one-way ANOVA was performed (*p<0.05). (CFT=clot formation time, Alpha angle, MCF=maximum clot firmness, ND=not detected)

FIG. 5—Promotion of clot formation in thrombocytopenic blood and trapping of platelets by the mesh-like structure of CellN90. (A) NATEM clotting profiles of blood from severely thrombocytopenic patients with and without addition of CellNF90 suspended in PBS (1 wt. %) (ND=not detectable). (B) FESEM images of the blood clot from Patient #3 with and without CellNF90. (C) Fluorescence images of washed platelets spread on a glass coverslip and a CellNF90-coated glass coverslip. The actin within the cytoskeleton of the platelets was stained using Alexa-488 Phalloidin (green).

FIG. 6—Lysis of red blood cells and fibrin formation induced by CellNF90 and a commercial hemostat, ORC. (A) Indirect lysis study: the lysis of red blood cells in contact with the supernatant of CellNF90 and ORC suspensions (pH of each supernatant is indicated). (B) Direct lysis study: the lysis of red blood cells in direct contact with freeze-dried CellNF90 and dry ORC. Lysis percentages were calculated using lysis of red blood cells in contact with deionised water (100% lysis) and PBS (0% lysis) as a positive and a negative control, respectively. Mean values (n=3) are shown with ±SE and standard one-way ANOVA was performed (*p<0.05 and **p<0.01). (C) FESEM images of red blood cells in contact with freeze-dried CellNF90 and dry ORC (direct lysis study). (D) A5 (amplitude of clot 5 min after onset of clotting time) of blood supplemented with fibrinogen (doses as indicated) in the absence (control containing PBS) and presence of CellNF90 and ORC (left axis), and expressed as a percentage of the A5 value of the control blood (right axis). Mean values (n=3-7) are shown with ±SE (standard error) and one-way ANOVA was performed (*p<0.05).

FIG. 7—(A) The amount of blood loss in a mice liver injury model with no haemostat applied (control), 15 mg of CellNFs applied (shown as “CNFs”) in dry sponge form, or the same weight of ORC applied. Mean values (n=10) are shown with ±SE (standard error) and one-way ANOVA was performed (*p<0.05). (B) FESEM images of the liver injury wound site with no haemostat applied (control), CellNFs applied (shown as “CNFs”) and ORC applied. Representative images of the excised liver at the termination of the experiment is also shown as inserts in the top-left corner of the FESEM images.

FIG. 8—(A) endothelial cell proliferation 3, 24 and 48 hours following addition of the supernatant from CellNFs (shown as “CNFs”), ORC or KG suspended in PBS for 24 hours, as determined using a standard MTT (3-[4,5-dimethylthiazol-2-yl]-2,5-iphenyltetrazoliumbromide) assay. PBS was used as positive control, and a well without cells but containing each haemostatic material was used as negative control. Mean values (n=9 from 3 different passages) are shown with ±SE and standard one-way ANOVA was performed (*p<0.05). (B) as in A but with fibroblasts instead of endothelial cells.

FIG. 9—Representative images of wound healing following subcutaneous implantation of haemostatic materials in mice: 1 day to 4 weeks. No redness or inflammation was observed in the wounds of the mice assigned to the control procedure or the CellNFs, (shown as “CNFs”). In these groups, the sutures were expelled from the wound during the second week as a result of complete wound healing. In comparison, the surgical site of the mice implanted with the commercial agent, ORC, was reddened and swollen (as shown using arrow) for up to three days post-surgery. The sutures took slightly longer in this group to be expelled from the wound. n=3 mice per experimental group.

FIG. 10—(A) The sedimentation value (Hₛ/Hᵢ) representative of fiber aspect ratio and BET specific surface area of chitin milled for 0 to 6 hours (chitin nanofibers; CTNF, with time expressed in hours of milling). (B) The rheological spectra of CTNF0, CTNF5 and CTNF6 subjected to an increasing oscillating strain (strain sweep) at a constant frequency. Elastic (G′) and viscose (G″) modulus are representative of solid-like and liquid-like behavior of viscoelastic materials, respectively. (C) FESEM images of the CTNF0, CTNF5, and CTNF6 on a silicon substrate (magnification: CTNF0: 5KX, CTNF5 and CTNF6: 100 KX).

FIG. 11—(A) Coagulation parameters obtained from NATEM assays in the presence of CTNFs with different milling times in comparison with chitosan (collected from Celox®) suspended in PBS (1 wt. %). Results are expressed as a percentage of the coagulation parameters obtained from the control sample consisting of blood and an equivalent volume of PBS. (B) Coagulation parameters obtained using the NAFibTEM assay relative to control blood in the presence of CTNF0, CTNF5, CTNF6, and the chitosan collected from Celox®, with the calculated contribution of plasma coagulation and platelet activation in A10. (C) As in A, except that CTNF5 is compared with Celox in a dry form at the same sample-to-blood weight ratio. The native blood was used as a control. Mean values (n=3-9) are shown with ±SE (standard error) and one-way ANOVA was performed (*p<0.05). (CT=clotting time, CFT=clot formation time, A10=clot amplitude at 10 mins and MCF=maximum clot firmness). (D) SEM images of the blood clot of CTNF5 (magnification: 5 KX).

FIG. 12—Clotting profiles obtained using the NATEM assay in the presence and absence of CTNF5 suspended in PBS at 1 wt. %, with (A) Platelet-poor plasma (PPP) (B) plasma deficient of coagulation Factor-IX, (C) plasma deficient of coagulation Factor-XI and (D) plasma deficient of coagulation Factor-XII.

FIG. 13—Clotting profiles obtained using the Intrinsic pathway activated ROTEM assay (InTEM) in the presence and absence of CTNF5 (A, B) and CellNF90 (shown as “CNF1.5”); C, D) suspended in PBS at 1 wt. %, with platelet-poor plasma (PPP) (A,C) and plasma deficient of coagulation Factor-XII (B,D).

FIG. 14—Summary of the coagulation mechanism induced by CellNF90. (A) Normal blood vessel with intact endothelium preventing interactions between platelets and subendothelial proteins. (B) Following injury, platelets are activated following interaction with components of the sub-endothelium. (C) The force of the blood flow from the damaged blood vessel and the extent of the injury prevents an adequate thrombus from forming. (D) CellNFs with a high aspect ratio act as a mesh-like physical barrier to trap platelets, red blood cells and bind fibrinogen. (E) CellNFs with a high specific surface area enhance the formation of an effective clot through activation of the intrinsic pathway of plasma coagulation, with the generation of thrombin promoting fibrin formation and platelet activation.

FIG. 15—Compositions of the invention comprising α-cellulose ball-milled for 90 minutes in two forms: (A) dispersed in PBS (1 wt. % gel); and (B) sponge freeze-dried form.

FIG. 16—Representative TEMograms obtained from the NATEM and NAFibTEM Assays. (A) Non-activated thromboelastometry (NATEM), wherein CaCl₂ is added to the blood and the kinetics of whole blood clot formation including the effect of both platelets and plasma is measured. (B) NAFibTEM assay (non-activated fibrin based thromboelastometry) in which CaCl₂ and cytochalasin-D are added, thus measuring plasma coagulation without platelet contribution to the clot.

### Definitions

The terms “aspect ratio” and “apparent aspect ratio” as used herein are interchangeable—dense meshed networks of certain fibres, such as cellulose fibres derived from lignocellulosic biomass, are not truly monofilament (ie. having clear and uniform fibre length or diameter), but complex branched or interlinked structures. The “aspect ratio” or “apparent aspect ratio” is calculated using sedimentation samples with different initial fibre concentrations as described in the Examples section.

As used herein the term “haemostasis”, refers to controlling, reducing or stopping bleeding from a surface and related terms such as “haemostatic” and “haemostat” have correspondingly similar meanings.

As used herein the term “mesh”, mesh-like” or related/derived terms relate to any interwoven, interlinked, entangled or otherwise intertwined 3-D networks of fibres, which may include a degree of branching, which branching linkages may, for example, comprise covalent or hydrogen bonds or involve Van-der-Waal interactions, or any combination thereof. For example, cellulose is a linear polymer, but glucose hydroxyls on one cellulose chain may form strong hydrogen bonds with annexed oxygens on glucose moieties of annexed cellulose chain(s).

A “therapeutically effective amount”, as referred to herein, includes a sufficient, but non-toxic amount of a compound or composition to provide the desired therapeutic effect. The “effective amount” will vary from subject to subject depending on one or more of a number of factors amongst, for example, the particular agent being administered, the severity of the condition being treated, the species being treated, the age and general condition of the subject and the mode of administration. For any given case, an appropriate “effective amount” may be determined by one of ordinary skill in the art using only routine experimentation. Typically, “therapeutically effective amount” in the context of the present invention refers to an amount sufficient to result in haemostasis, and therefore recession/reduction, inhibition, or cessation of bleeding.

### DETAILED DESCRIPTION OF THE INVENTION

Hemorrhage, or severe blood loss, be it via serious trauma, childbirth, complex surgery, drugs, disease or genetic disorders, accounts for a large number of deaths. Half of trauma and combat deaths occur as a result of uncontrolled hemorrhage in the first hours of injury before reaching a treatment facility. Those who reach hospital facilities remain in danger of early mortality through continued hemorrhage and coagulopathy.

A wide range of haemostatic agents have been developed, some targeting coagulation/clotting pathways and others acting through occlusion and/or absorption/adsorption of fluids, and are composed of materials such as fibrin-based glues or sealants, zeolite powders, kaolin-impregnated gauze (KG), gelatin foams and oxidized regenerated cellulose (ORC) films. However, each of these materials has associated drawbacks, including acidity causing burns, bystander cell damage and/or inflammation, non-biocompatibility and/or no-bioabsorbability, high cost and short shelf-life.

The current knowledge in cellulose-based haemostatic materials is that the mechanism of cellulose to work as a haemostatic material is by absorbing water and in turn by thickening blood. In addition, it is widely believed that, in order to give additional heamostatic function to cellulose, oxidation of cellulose is necessary. However, the present studies have shown that nano-scale 3D-meshed networks of non-oxidised cellulose fibres (CellNFs) can enhance haemostasis. Without wishing to be bound by theory, it is considered that the meshed networks of the invention may enhance haemostasis as follows: (i) negatively-charged CellNFs with high specific surface area enhance induction of the intrinsic pathway of plasma coagulation resulting in thrombin generation; (ii) CellNFs with high specific surface area adsorb fibrinogen within the plasma thus promoting fibrin strand formation through the actions of thrombin; and (iii) CellNFs with high aspect ratio form mesh-like physical barriers against, trapping and concentrating, platelets at the location where thrombin and fibrin strands are also focused. The combination of these effects results in the rapid formation of a robust clot for not only healthy patients but also thrombocytopenic and heparinized patients.

Systematic studies were undertaken to determine the relationship between the physical properties of CellNFs and their hemostatic behavior. CellNFs, when optimized for specific surface area and fibre aspect ratio, shortened clotting time by 68% (±SE 2%) out-performing the most commonly-used hemostats. Further studies confirmed the principal pro-coagulant activity of CellNFs occurs through the induction of the intrinsic pathway, resulting in robust fibrin clot formation and, consequently, the activation of platelets trapped in the mesh-like structure. CellNFs were also found to enhance clotting in platelet-deficient and heparinized blood. In contrast to commercial cellulose-based hemostats, CellNFs are not acidic and, hence, did not impede fibrin formation nor did they induce bystander cell damage.

The present studies showed that there is a ‘sweet spot’ in the dimensions of a nano-scale 3D meshed material for enhanced haemostatic function: the fibre-component should have mean diameters lower than 1 μm and an aspect ratio (ratio of fibre length to diameter) of at least 100, and the meshed network should have a specific surface area of at least 10 m²/g, and a gel point (which reflects the interconnectivity/meshing degree of the fibres in the network) no greater than 3 g/L. More specifically, a certain range of specific surface areas of the 3D-mesh structure is influenced by the combination of fibre diameter and fibre length, or its technically equivalent combination of fibre diameter and fibre aspect ratio.

These materials provide one or more of the following advantages at the same time without further chemical or physical modifications and addition of extra reagents; (1) improved clotting time, including with patients with clotting disorders or who are taking anticoagulant therapy; (2) activation of fibrinogen polymerisation, (3) entrapment of platelets so as to enhance their activation, (4) absence of hemolysis, inflammation, burns and, in particular embodiments, (5) bioabsorbable/biocompatible nature.

### Fibrous Materials

Any suitable non-acidic fibrous material as known in the art, provided it is not toxic to animals, may be used, noting the need for the fibre-component to have mean diameters lower than 1 μm and an aspect ratio (ratio of fibre length to diameter) of at least 100, and for the meshed network to have a specific surface area of at least 10 m²/g, and a gel point no greater than 3 g/L. Such fibrous material may be of biological origin, or be derived therefrom, or be artificial, and may include a wide variety of polymeric materials, including, for example, collagen, calcium alginate, chitin or chitosan, polyester, polypropylene, polysaccharides, polyamines, polyimines, polyamides, polyesters, polyethers, polynucleotides, polypeptides, proteins, poly (alkylene oxide), polyalkylenes, polythioesters, polythioethers, polyvinyls, polymers comprising lipids, and mixtures thereof.

According to certain embodiments the non-acidic fibrous material is branched, via covalent bonds, hydrogen bonds or Van-der-Waal interactions, or any combination thereof. An example of a branched material which does not include covalent linking is cellulose, where chains are linear and unbranched in a strict chemical sense, but which may form hydrogen bonds with one-another, resulting in macrofibres with what is a physically, if not chemically branched structure. However, the present invention also contemplates the use of materials which include chemical branching such as, for example, a material comprising a β1→4 backbone with β1→3 and/or β1→6 branching linkages. Alternatively, the material may include polymers which are synthesized with an adequate concentration of cross-linking agent to provide the desired level of cross-linking during polymerisation (see, for example, polyacrylamides), or polymers which are cross-linked when already formed—for example, linear monofilamentous polymeric chains cross-linked with one-another by appropriate cross-linking agents as known in the art.

According to particular embodiments, the fibrous material is of biological origin or is derived therefrom, and is biocompatible/bioabsorbable. According to certain embodiments, the fibrous material comprises collagen, calcium alginate, chitin, polylactate, polysaccharides, polynucleotides, polypeptides, proteins.

According to certain embodiments, the fibrous material comprises a non-oxidised polysaccharide. The polysaccharide may be selected from any suitable large, high molecular weight (high degree of polymerization) polysaccharide, and may be selected from, for example, cellulose or other β-glucan (which may be based on linear or branched β1→3, β1→4, β1→6 linkages, or mixtures thereof), chitin/chitosan, (β-mannan, glucomannan, galactomannan, galactoglucomannan, other polysaccharide based on a β-linked backbone, and starches (native or modified), or any combination thereof (many polysaccharides co-exist in natural sources; for example, woody biomass comprises a complex interlinked network of cellulose, hemicelluloses and lignin, and yeast cell walls comprise β1→3, β1→6 linked glucan with interspersed chitin).

According to a specific embodiment, the fibrous material is cellulose, including cellulose derived from biomass, such as any plant biomass, especially lignocellulosic biomass, or even electrospun cellulose.

The main chemical constituent of plant cell walls is cellulose, which is a non-branched polysaccharide polymer made up of glucose units. In wood fibres, cellulose is part of a complex interconnected network with hemicelluloses and lignin, in which the cellulose chain may have an average length of 5 μm corresponding to a degree of polymerization (i.e., glucose units) of 10,000, arranged in parallel to form bundles, called microfibrils, which have highly ordered crystalline regions (which impart strength) and disordered or amorphous regions (which impart flexibility). In addition to the organization of the microfibrils, the structural complexity of the cell wall is increased by being organized into a number of layers differing by the angle of the cellulose microfibrils to the longitudinal fibre axis. Within the aggregation of ordered and disordered regions, cellulose fibres in lignocellulosic biomass range from 3 to 100 μm of size in diameter and 1-4 mm in length. In order to obtain a meshed network of fibres with a high aspect ratio, high specific surface area, high level of interconnectivity and low gel point, there is a need to break open the macrofibres.

Agricultural wastes, including lignocellulosic biomass waste from wood milling or pulping or bagasse from sugar cane waste, are environmentally attractive and inexpensive sources of macrofibrous cellulose. Moreover, the efficient use of the agricultural wastes is good for the environment.

Lignocellulosic biomass consists of cellulose and a number of other materials such as lignin and hemicelluloses as well as pectins, waxes. The pretreatment of agricultural wastes, such as lignocellulosic biomass, to at least enrich the cellulose content is therefore highly desirable, if not necessary. Typical pretreatment processes include acid-chlorite treatment (delignification/bleaching) and alkaline treatment.

Delignification/bleaching, a well-known process in the art, removes most of lignin and other components through the combination of sodium chlorite and acetic acid (fed into the reactor at regular intervals) stirred into the lignocellulosic biomass at 70-80° C. over a period of 4-12 hours followed by stirring for several hours before washing with distilled water until reaching neutral pH. The obtained solid products are collected and dried and mostly includes hemicellulose and cellulose in the fibres.

Alkaline treatment, typically using sodium hydroxide, removes the hemicellulose fraction and remaining lignin, and is also well known in the art. Once thoroughly washed to neutrality and dried, the obtained fibre product is mostly cellulose, with most non-cellulosic materials removed.

Other processes, including enzymic, or enzyme-assisted processes, or modified versions of the acid-chlorite and alkaline treatments for enriching or purifying cellulosic fibres from cellulosic biomass are also known in the art and are also contemplated within the scope of the present invention.

### Processes for Preparing Meshed Cellulose Networks from Biomass

Once biomass is at least partially fractionated to at least enrich, if not purify the cellulose, or even before such fractionation, it may be treated to at least partially break down the macro-fibrous and organized/crystalline structure in favour of cellulose fibrils.

Several techniques have been developed for the extraction of fibrillated cellulose from cellulosic materials. The different extraction methods result in differences in types and properties of the obtained nanocellulose and include acid hydrolysis, enzymatic hydrolysis and mechanical processing. Acid hydrolysis results in drawbacks such as acid wastewater, potential opening of ring structures in the cellulose, resulting in acidic groups, and incomplete neutralization of the product. Enzymatic hydrolysis, while being mild and not resulting in the drawbacks of acid hydrolysis, is comparatively very slow, may require the use of other techniques and/or enzymes and is significantly more expensive. Mechanical processing is relatively well understood, relatively quick and inexpensive, and does not require disposal or neutralization of waste streams or the product itself. Furthermore, mechanical processing allows for simpler control of processing and degree of fibrillation of cellulose. Cellulose chains are unbranched, but they form hydrogen bonds with adjacent chains, forming fibrils, which in turn interact with one another to form larger cellulose macrofibres with diameters of several hundred nanometers to micrometres, and lengths of micrometres. Mechanical processing according to the present invention results in at least partial disruption and opening up of such macrofibres, creating a ‘branched’ and mesh-like structure. Thus, according to an embodiment of the present invention, a meshed network of branched cellulose fibrils with low mean diameter, high aspect ratio, high specific area and low gel point is obtained by mechanical processing of a high molecular weight cellulose or material containing high molecular weight/macrofibrous cellulose such as at least partially fractionated lignocellulosic biomass. Such a process requires no chemical treatment and no-regeneration, thus minimising chemical contamination and possible reduction of production costs.

Mechanical processing involves the isolation of cellulose fibrils by applying high shear force, such as by high pressure homogenization, ultrasonication or ball-milling to cleave the cellulose fibres along their longitudinal axes.

High pressure homogenization involves applying high pressure and/or velocity to cellulose slurry, in some cases forcing material through small orifices under pressure, resulting in cleavage of the cellulose microfibrils, and can result in fibre diameters of 10-20 nm with lower crystallinity than the original cellulose. The pressure and speed applied during homogenization can be adjusted as desired, and by no more than routine experimentation, depending on the source cellulosic material and final mean fibre diameter, aspect ratio, specific area and gel point desired.

Ultrasonication creates localized areas of cavitation resulting in very high shear forces as well as localized high temperatures and can result in cellulose fibres with a diameter of 10-100 nm. The power and amplitude of the ultrasonic energy, and exposure time can be adjusted as desired, and by no more than routine experimentation, depending on the source cellulosic material and final mean fibre diameter, aspect ratio, specific area and gel point desired.

Ball milling is a comparatively simple mechanical method, which operates through shear forces and impacts created among and between milling balls (made of, for example, zirconium, steel, titanium) or grinding media and the surface of the vessel. Although generally used for grinding/pulverising materials, by controlling the milling conditions and times by the methods of the present invention, the present studies have shown that ball-milling can be used for controlled defibrillation of cellulose fibrils, and may also be used for direct extraction of fibrillated cellulose from cellulosic biomass (that is, without the need for pretreatment of the biomass by bleaching and/or alkaline treatment). In the course of the present studies it has been found that controlled ball-milling of macrofibrous cellulose successfully opens up the structure of the macrofibres, resulting in an intermeshed, cross-linked or branched structure with a low mean fibre diameter, a high fibre aspect ratio (mean fibre length/mean fibre diameter), a high specific surface area, and a low gel point.

Thus, according to an embodiment of the present invention, a meshed network of cellulose fibres with low mean diameter, high aspect ratio, high specific area and low gel point is obtained by ball milling.

There are various types of ball mills, including the planetary ball mill, mixer ball mill, tumbler ball mill, and vibration ball mill, with the planetary ball mill being commonly used in industry.

There are various variable factors which affect the characteristics of ball-milled products such as, the number and size of balls, the milling speed, the state of milling (dry or wet state), the concentration of material suspended in the milling fluid (often water), the weight ratio between balls and materials, and milling time.

Dry milling, while still contemplated within the scope of the present invention, can result in aggregation of materials and inconsistent or non-uniform milling of materials. Thus, in an embodiment of the present invention a meshed network of cellulose fibres with low mean diameter, high aspect ratio, high specific area and low gel point is obtained by wet ball-milling

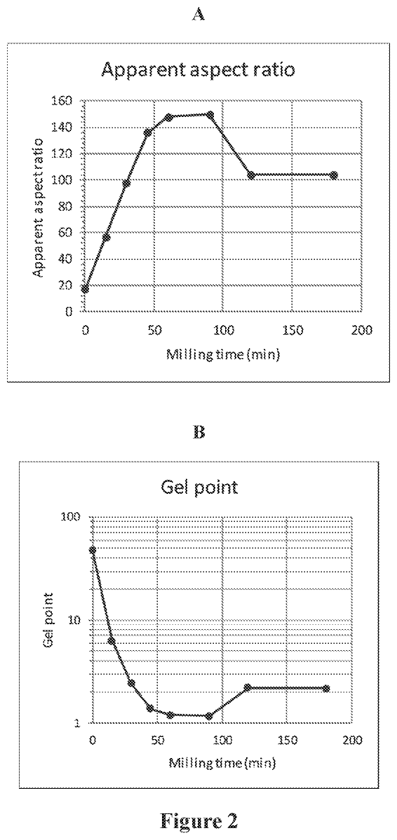

The number and size of balls, the milling speed, the weight ratio between balls and materials, fibre concentration, and milling time can all be adjusted as desired, by no more than routine experimentation, depending on the source cellulosic material and final mean fibre diameter, aspect ratio, specific area and gel point desired. Notably, there will be an optimum range for each of these parameters, as a minimum amount of milling will be required to achieve the desired final mean fibre diameter, aspect ratio, specific area and gel point, but there will also be a point after which the cellulose fibrils are broken up excessively, resulting in an ever-increasing specific surface area, but the aspect ratio will reach a peak and then decline with ever-decreasing mean fibre length (as shown in FIG. 2A), and the gel point will also reach a lowest point and then increase (as shown in FIG. 2B).

### Meshed Networks of Fibres

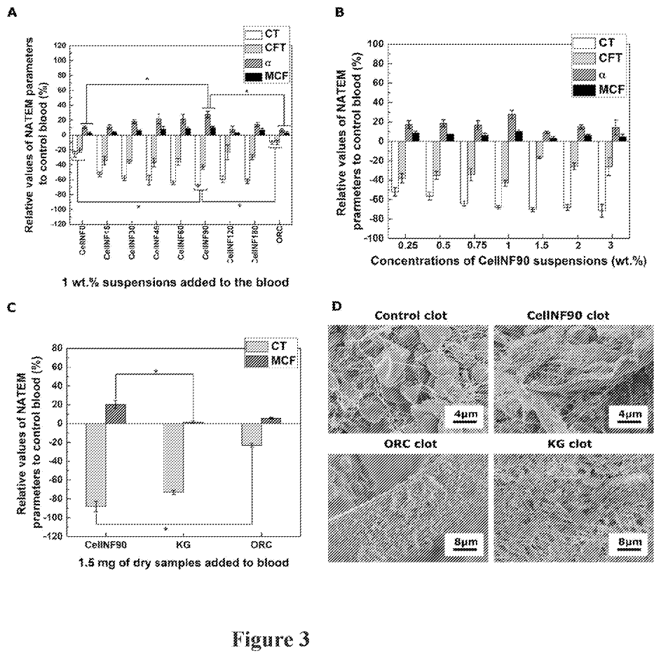

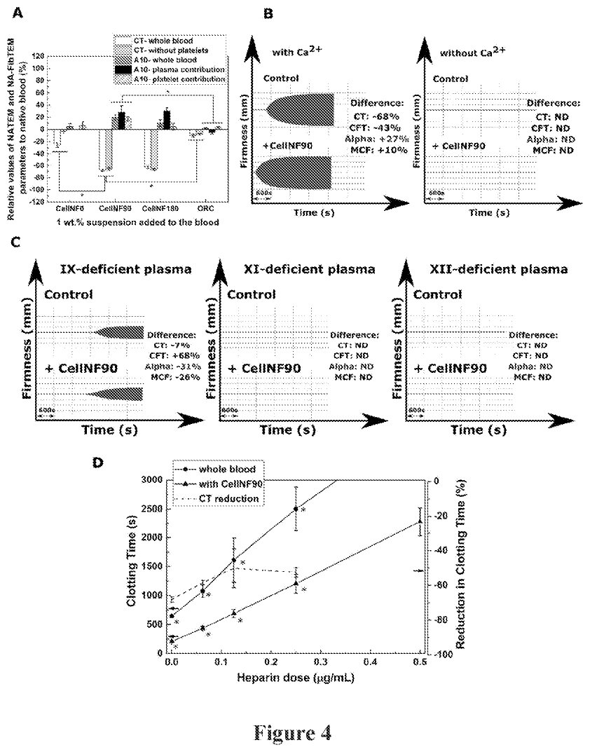

The present studies have shown that platelet activity induced by milled fibres, has a strong correlation with the change in aspect ratio as a function of milling time (FIG. 3). Likewise, the present studies have shown that plasma-related coagulation induced by milled fibres has strong correlation with the change in specific surface area as a function of milling time (FIG. 4).

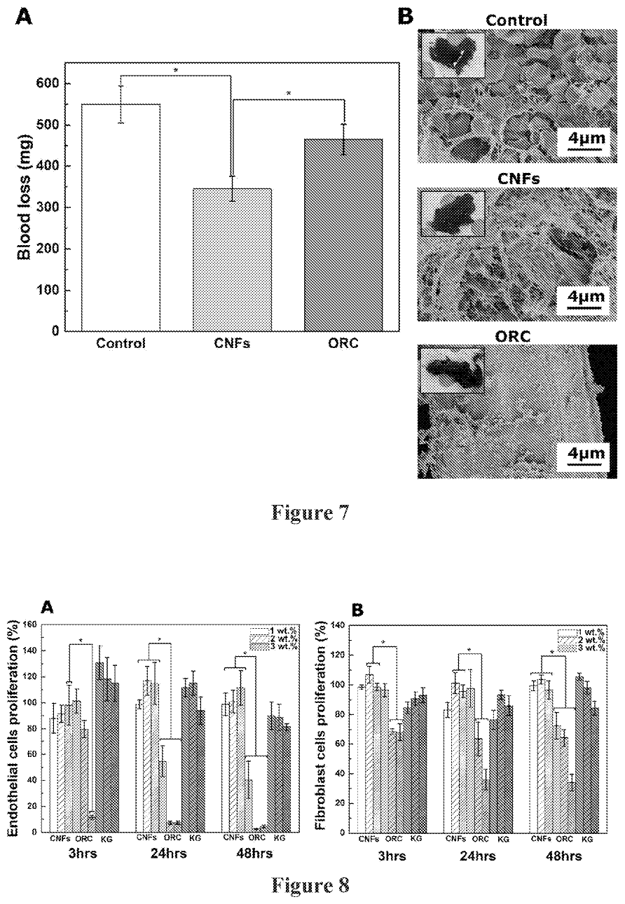

Without wishing to be bound by theory, it is believed that a fine meshed network of fibres mediates blood coagulation through a combination of three main mechanisms: (i) negatively-charged CellNFs with high specific surface area enhance induction of the intrinsic pathway of plasma coagulation resulting in thrombin generation (ii) CellNFs with high specific surface area adsorb fibrinogen within the plasma thus promoting fibrin strand formation through the actions of thrombin (iii) CellNFs with high aspect ratio form mesh-like physical barriers against, trapping and concentrating, platelets at the location where thrombin and fibrin strands are also focused (FIGS. 7D and 7E). The combination of these effects results in the rapid formation of a robust clot in the blood from not only healthy patients with/without heparin added, but also from severely thrombocytopenic patients. As the positive domain of fibrinogen can cause irreversible adsorption on negatively charged surfaces, the larger specific surface area of CellNF90 leads to adsorption of nano-sized plasma proteins such as fibrinogen (475 Å) and induces more rapid fibrin formation. We speculate that CellNF90-enhanced fibrin formation is a result of increased thrombin production following the induction of plasma coagulation. The production of thrombin also results in the activation of platelets thus enabling the platelet receptor, αIIbβ3, to bind to the fibrin strands.

It was unexpectedly discovered that these key features, which are not present in non-defibrillated conventional micron-scale fibres, can be realized by changing the dimension and branched structure of cellulose fibres without altering surface chemistry. It was also unexpectedly discovered that a ‘sweet spot’ of optimized aspect ratio and specific surface area can be readily realized by ball milling in the case of cellulose. It was also unexpectedly discovered that the combination of these features results in successful and rapid clotting of blood in the blood of patients with thrombocytopenia or healthy blood of patients supplemented with heparin.

Thus, non-acidic meshed networks of fibres for use in the compositions and methods of the present invention comprise fibres with a low mean diameter, of no greater than 1 μm, a high aspect ratio (mean fibre length/mean fibre diameter) of at least 100, and the meshed network of fibrous material has a specific surface area of at least 10 m²/g, and a gel point no greater than 3 g/L.

Meshed networks for use in materials according to the invention may also be prepared using polymeric materials other than cellulose, provided they have the necessary minimal fibre aspect ratio, specific surface area and gel point. While high molecular weight ‘branched’ cellulosic materials as described in the examples are ideal according to the present invention, other materials may are also contemplated, such as inherently branched/cross-linked materials or cross-linked monofilamentous materials, as briefly described earlier—once given the present teachings a person skilled in the art would be able to determine suitable polymeric materials and appropriate cross-linking agents and concentrations thereof by no more than routine trial and experimentation.

In certain embodiments, mean fibre diameters may be between 500 nm and 1 nm, such as between 200 nm and 5 nm, between 100 nm and 5 nm, between 70 nm and 5 nm, between 50 nm and 5 nm, between 40 nm and 5 nm, between 30 nm and 5 nm, between 20 nm and 5 nm, about 10 nm, about 20 nm, about 30 nm, about 40 nm or about 50 nm.

In certain embodiments, the aspect ratio may be between 100 and 180, such as between 110 and 170, between 120 and 170, between 130 and 170, between 140 and 170 between 150 and 170, about 170, about 160, about 150, about 140, about 130 or about 120.

In certain embodiments, specific surface areas of the meshed network may be between 10 m²/g and 20 m²/g, such as between 12 m²/g and 19 m²/g, between 13 m²/g and 18 m²/g, between 14 m²/g and 17 m²/g, between 15 m²/g and 17 m²/g, between 16 m²/g and 17 m²/g, about 13 m²/g, about 14 m²/g, about 15 m²/g, about 16 m²/g, or about 17 m²/g.

According to certain embodiments the meshed network comprises fibres with a mean diameter between 100 nm and 10 nm, an aspect ratio of at least 120, and has a specific surface area of at least 13 m²/g, and a gel point no greater than 1.5 g/L.

According to certain embodiments the meshed network comprises fibres with a mean diameter between 50 nm and 5 nm, an aspect ratio of between 150 and 170, and has a specific surface area of between 15 m²/g and 17 m²/g, and a gel point of between 1.1 g/L and 1.3 g/L.

According to specific embodiments the meshed network comprises cellulose fibres with a mean diameter between 100 nm and 10 nm, an aspect ratio of at least 120, and has a specific surface area of at least 13 m²/g, and a gel point no greater than 1.5 g/L.

According to further specific embodiments the meshed network comprises cellulose fibres with a mean diameter between 50 nm and 5 nm, an aspect ratio of between 150 and 170, and has a specific surface area of between 15 m²/g and 17 m²/g, and a gel point of between 1.1 g/L and 1.3 g/L.

According to yet further embodiments, the meshed network comprises cellulose fibres with a mean diameter between 50 nm and 5 nm, an aspect ratio of between 160 and 170, and has a specific surface area of between 16 m²/g and 17 m²/g, and a gel point of between 1.1 g/L and 1.2 g/L.

### Haemostatic Compositions

Compositions for controlling bleeding may be formulated using non-acidic meshed networks of fibrous material according to the present invention and will typically be prepared by methods known to those of ordinary skill in the art. The compositions may be administered by standard routes for bleeding wound treatment. For example, the compositions may be applied directly to an open wound or bleeding surface by topically or intracavitary routes.

In certain embodiments, the meshed network of fibres may be the sole component (other than water or buffered/isotonic solution) of the composition, being formulated into a powder, a gel, a film, a composite spray, a dressing (including gauzes, webs, pads, tampons, foams, tapes), particles, pellets, sponges, plugs and the like using well established techniques in the art.

Powders as well as some particles and pellets may be prepared by freeze-drying of the meshed networks obtained as described above, optionally after further purification and, in the case of particles and pellets, a forming step.

Other compositions of the present invention may include excipients such as a pharmaceutically or veterinary acceptable carriers, diluents binders and/or adjuvants and surfactants (as wetting agents) as known on the art, or combinations thereof.

Non-acidic meshed networks of fibres according to the present invention may also be incorporated, optionally along with other active agents, into or onto biodegradable or non-biodegradable polymers as known in the art as carriers or forming structures/agents (as, for example, scaffolding, backing tape, binding agent or the like). According to one embodiment, the carrier layer comprises a viscose non-woven material, or alternatively it may comprise a woven gauze, a film, a foam, or a sheet gel. The material of the carrier material may or may not be degradable in conditions associated with wounds in or on a human or animal body and may be, for example, polyester, polypropylene, acrylic or polyethylene based. However, at least for intracavitary administration routes, any supporting/carrier structures (such as webs, plugs, tapes and the like) or binders would beneficially be made of biodegradable polymers, such as polylactic acid, polylactides, poly(lactic-co-glycolic) acid, poly(caprolactone), polyglycolides, polyhydroxybutyrate, chitosan, hyaluronic acid, modified celluloses, unmodified and modified starches.

According to one aspect of the invention, the meshed network of fibres may be bonded to a carrier layer, optionally both sides of a carrier layer, using heat and/or pressure or may be bound there using a pharmaceutically acceptable adhesive or through flash-freezing following by lyophilization.

In certain embodiments, there may be a further layer on top of the meshed network layer which is bonded to the carrier layer by heat and/or pressure, the further layer comprising a soluble, dispersible or removable retaining material which may be peeled off, or dissolved or degraded by, or dispersed in, bodily fluids when the composition is applied to a wound. This further layer can also be used to retain the meshed network of fibres and may be a soluble film made from a biodegradable or biocompatible material such as, for example, gelatine or a cellulose derivative, or it may be made from a soluble film-former such as polyvinyl acetate (PVA) or polyvinyl alcohol (PVOH).

According to another embodiment, the binding to a carrier layer is chosen so that even when it is wetted with blood, at least a portion of the composition will remain in an area of bleeding even when the carrier layer is removed. Previously developed haemostatic materials do not leave any haemostat at the wound site once the material is removed, so bleeding resumes.

This can be achieved by having the meshed network bonded so that it is released from the bonding layer when it gets wet. This is effective if the binding is sufficiently water sensitive to weaken the bond when the combination is wet.

The compositions of the invention may take any suitable form and may be provided in a range of different sizes, shapes and thicknesses necessary to deal with a wound, such as square, rectangular, circular or elliptical. For example, the material may be a generally flat shape with little height relative to its width/depth. Any regular or irregular shape may be employed. It may be provided in large sheets which can be cut to the required size.

In certain embodiments of the invention, the compositions according to the invention may further include haemostatic agents, or other biological or therapeutic agents, moieties or species, including drugs and pharmaceutical agents. The agents may be bound within the polymeric matrix, as well as to the fabric surfaces and/or within the fabric. The agents may be bound by chemical or physical means (such as through lyophilisation). The agents may be dispersed partially or homogenously through the fabric and/or the polymeric matrix. For example, the agent(s) may be covalently linked to the fibres, such as by a reversible imine bond between hydroxyls of sugar moieties and amine groups of proteins/peptides (optionally backed up by, for example, further reaction with a reducing agent such as sodium borohydride or sodium cyanoborohydride to form an irreversible secondary amine linkage and thereby provide a stronger binding).

Preferred biologics, drugs and agents include haemostatic agents, analgesics, anti-infective agents, antibiotics, adhesion preventive agents, pro-coagulants, and wound healing growth factors.

Haemostatic agents that may be used in compositions according to the invention include, for example, therapeutically effective proteins (including procoagulant enzymes) and peptides selected from the group consisting of prothrombin, thrombin, fibrinogen, fibrin, fibronectin, heparinase, Factor X/Xa, Factor VII/VIIa, Factor IX/IXa, Factor XI/XIa, Factor XII/XIIa, tissue factor, batroxobin, ancrod, ecarin, von Willebrand Factor, collagen, elastin, albumin, gelatin, platelet surface glycoproteins, vasopressin and vasopressin analogs, epinephrine, selectin, procoagulant venom, plasminogen activator inhibitor, platelet activating agents, synthetic peptides having hemostatic activity, derivatives of the above and any combination thereof. In particular embodiments, the haemostatic agents are thrombin, fibrinogen, fibrin or combinations thereof.

Where the material is provided in a sterile form, sterilisation may be carried out using any of the conventionally known methods, such as gamma irradiation, electron beam treatment, heat treatment (autoclaving), etc. A material in a non-sterile form may be provided in combination with one or more preservatives.

### Methods for Controlling Bleeding

The present invention also relates to methods for controlling, reducing or stopping blood flow from a bleeding body surface, which may be internal or external, in any animal, but particularly contemplated patients are any mammals, and especially humans. The methods may comprise applying to the surface of concern a composition according to the invention, optionally comprising the step of cleaning a wound area where necessary and/or possible prior to applying the composition, and/or the step of applying constant pressure to the wound area after applying the composition, until clotting occurs.

According to a related aspect of the present invention, there is provided the use of a composition according to the invention for the manufacture of a medicament for controlling bleeding of an animal. For example, sponge samples can be inserted into internal wounds using an applicator and, in the case of other wounds, they can be pressed on the wound site until hemostasis is achieved.

Preferred forms of the present invention will now be described, by way of example only, with reference to the following examples, including comparative data, and which are not to be taken to be limiting to the scope or spirit of the invention in any way.

### EXAMPLES

### Example 1—Materials and Methods

### Synthesis of Cellulose Nanofibres

α-Cellulose powder (Sigma-Aldrich, Missouri, United States) was suspended in deionized water at a concentration of 0.5 wt. %. Cerium-doped zirconia balls with a diameter of 0.4-0.6 mm (Zirconox®, Jyoti Ceramic, Maharashtra, India) were then added to the suspension with a ball-to-powder mass ratio of 80. The mixture was milled using a Spex 8000M Mixer/Mill with milling times of 0, 15, 30, 45, 60, 90, 120 and 180 min. The samples were denoted according to the milling time, as CellNF0, CellNF15, CellNF30, CellNF45, CellNF60, CellNF90, CellNF120, and CellNF180. The synthesized CellNFs were compared with equivalent weights or concentrations of commercial hemostats made of ORC (Surgicel Fibrillar®, Johnson and Johnson, New Jersey, United States) and kaolin-impregnated gauze (QuickClot Combat Gauze®, Z-Medica, Connecticut, United States).

### Physicochemical Characterization

Fourier transform infrared (FTIR) spectra were obtained from freeze-dried samples using a Perkin Elmer Lambda spectrometer in a range between 500 and 4000 cm⁻¹ at a resolution of 4 cm⁻¹.

The crystal phases of the pressed freeze-dried samples were studied by X-ray diffractometry (XRD) using a Bruker D2 Phaser X-ray diffraction instrument at 30 kV and 10 mA, in a diffraction angle range from 10° to 60° with a Cu Kα radiation (λ=1.542 Å)). The degree of crystallinity, Dcr, was estimated using the Segal's method:

$$Dcr = ( \frac{I max - I min}{I max} ) \times 100$$

where Imax is intensity of the diffraction peak at 22° corresponding to (110) lattice diffraction and Imin is the height of the minimum in diffraction intensity around 18° between the (100) and (010) interferences and (110) lattice diffraction.

The average aspect ratio (length to diameter) of fibres was estimated using a sedimentation technique. Sedimentation tests were carried out in a cylinder using 25 mL of CellNF suspensions in deionized water, at solids concentrations ranging from 0.05 wt % to 3 wt %. The suspensions were first agitated for 2 min and then allowed to settle for a minimum of 48 hours. Once the suspension had settled completely, the ratio between the sediment height (hg) to the initial height of suspension (hᵢ) in the cylinder was measured. A quadratic equation was fitted to the graph of initial solid concentration (kg/m³) versus the ratio of sediment height to initial suspension height (hₛ/hᵢ). The linear term of the fit then gives the gel point solids concentration, G. Aspect ratio was calculated based on Crowding Numbers Theory as 5.66×(G×10⁻³)^{-0.5} with an assumption that all samples have the maximum water adsorption (0.5 g water/g fibre), so that the effective density of swollen cellulose nanofibres is 1333 kg/m³.

The specific surface area of freeze-dried samples was measured by the Brunauer-Emmett-Teller (BET) N₂ adsorption method using a Micromeritics Tristar 11-3020 instrument. Prior to the BET measurements, the samples were degassed at 115° C. for 4 hours.

Scanning electron microscopy (SEM) was employed to study the size and morphology of the CellNFs, using a Zeiss UltraPlus analytical field emission scanning electron microscope (FESEM) operated at 2 kV. SEM samples were first dispersed in DI water (0.01 wt. %) and the one drop of the dispersion was dried on a silicon substrate. They were sputter-coated with platinum (Pt) prior to SEM examination.

### Haemostatic Behaviour Studies

The hemostatic behavior of CellNFs was evaluated using whole-blood viscoelastic testing using a ROTEM-Delta machine (Pentapharm GmbH, Munich, Germany) Blood samples were collected from healthy volunteers and severely thrombocytopenic patients who were not taking drugs or natural products reported to alter blood coagulation. Blood was collected into sodium citrated vacutainers and analyzed within 3 hours of collection.

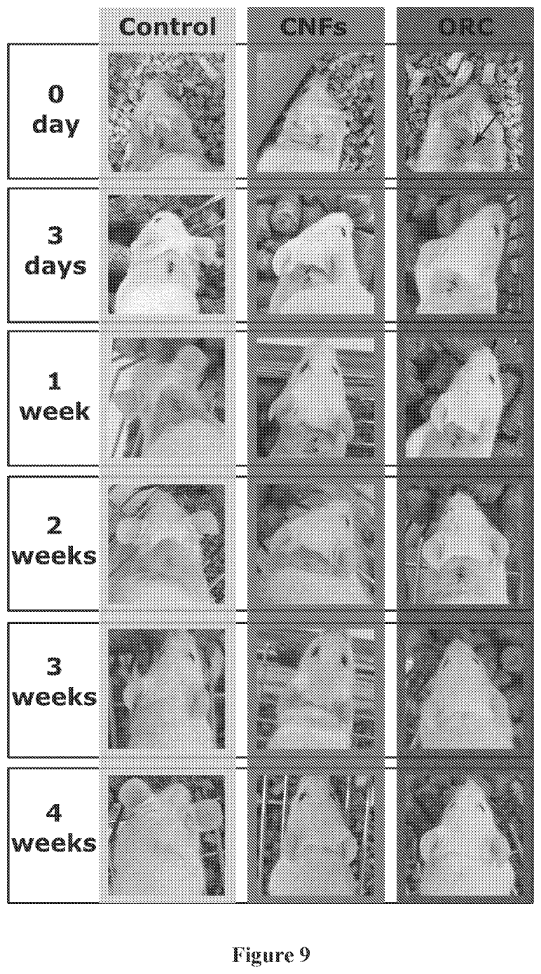

Two modes of ROTEM assays were used: one was non-activated thromboelastometry (NATEM), wherein CaCl₂ is added (Star-TEM reagent; Pentapharm GmbH, Munich, Germany) to the blood to study the kinetics of whole blood clot formation, including the effects of both platelets and plasma. The other was a modified FibTEM assay (NAFibTEM) in which CaCl₂ was added in combination with cytochalasin-D to neutralize the platelets (FibTEM reagent; Pentapharm GmbH, Munich, Germany) thus measuring the non-activated plasma coagulation process (FIG. 9). To perform the assays, haemostat samples were prepared at certain concentrations in PBS (1× pH 7.4), and then 50 μL of haemostat preparations were combined with 300 μL blood in Eppendorf tubes for 60 sec. Subsequently the mixture was transferred to the ROTEM cup with 20 μL of star-TEM or FiBTEM reagent and the assay immediately commenced. Control samples supplemented with an equivalent volume of PBS were run within each assay of 4 samples.

To study the dosage effect of CellNFs on coagulation properties, concentrations of 0.25, 0.5, 0.75, 1, 1.5, 2, and 3 wt % of the optimized sample were also studied. Preparations of commercialized ORC product were also tested.

ROTEM assays were also performed on the blood without the addition of CaCl₂ to demonstrate the effect of CellNFs on the induction of coagulation pathways rather than just absorbing the fluid and creating pin resistance.

In another test, the freeze-dried form of optimum CellNFs, dried ORC and dried kaolin-impregnated gauze (KG) were added directly to the ROTEM cup, the blood was then added on top immediately prior to the star-TEM reagent and the clotting profiles studied.

The ROTEM results of the control sample was used as a baseline for the ROTEM results of the experimental samples within each assay. Clotting time (CT), clot formation time (CFT), alpha angle (α) and maximum clot firmness (MCF) of CellNFs preparations were measured. As the NATEM assay measures whole blood clot formation whereas the NAFibTEM measures just fibrin clot formation, the contribution of plasma components and platelets to the clot can be determined by comparing the clot amplitude parameter, A10 (the amplitude of the clot 10 min after the onset of clotting), between the NAFibTEM and NATEM assays performed with the same experimental conditions. Calculations of the haemostatic effects of CellNFs and ORC sample (ORC) and control (C), on the contribution of platelets and fibrinogen to clot firmness were performed using the following equations:

$$A 10 ( Whole Blood ) = ( \frac{A 10 N ( S ) - A 10 N ( C )}{A 10 N ( C )} ) \times 100$$

$$A 10 ( fibrinogen contribution ) = ( \frac{A 10 F ( S ) - A 10 F ( C )}{A 10 F ( C )} ) \times 100 ,$$

$$A 10 ( platelets contribution ) = ( \frac{\begin{matrix}( A 10 N ( S ) - A 10 F ( S ) ) - \\ ( A 10 N ( C ) - A 10 F ( C ) )\end{matrix}}{( A 10 N ( C ) - A 10 F ( C ) )} ) \times 100$$

Where A10N is the A10 value of NATEM assay and A10F is the A10 value of NAFibTEM assay.

To evaluate fibrin formation induced by CellNF90 and compare it with that of ORC, recombinant fibrinogen (CSL-Behring, Pennsylvania, United States) was added to healthy donors' blood at dosages of 1, 2, 4, and 8 mg per mL, and NATEM assays were performed in the presence and absence of ORC and CellNF90 (in a 1 wt. % suspension). The relative change of the A5 parameter (the amplitude of the clot 5 min after the start of CT) to the control (PBS) was calculated.

In order to clarify the working mechanism of CellNFs, donor blood was heparinized using DBL heparin sodium (Hospira, IL, USA) with 0.0625, 0.125, 0.25 and 0.5 μg per mL of blood. The clotting time was then determined using the NATEM assay in the presence and absence of CellNFs.

To further clarify the effect of CellNFs on the induction of coagulation pathways, NATEM assays were performed wherein whole blood was substituted with an equivalent volume of Factor IX, XI, or XII deficient plasma (Instrumentation laboratory, Massachusetts, United States) in the presence and absence of CellNFs and analyzed on the ROTEM.

Blood samples from severely thrombocytopenic patients (reduced platelet counts) were studied using NATEM, to clarify the contribution of platelets in the mechanism of CellNFs.

### SEM of Clots

In order to visualize the interaction between blood and CellNFs, SEM images of the clots obtained at the completion of the ROTEM assays were taken using a Zeiss UltraPlus FESEM. The blood clots were fixed with 2% glutaraldehyde in a PBS buffer for 2 hours at room temperature, the clots were washed with PBS, deionized water then ethanol, then dried using a super critical dryer (40° C., 90 bar; Balzers CPD 030). The dried samples were sputter coated with Platinum prior to SEM imaging.

### Platelet Studies

The activation of platelets on optimized CellNFs was imaged using Brightfield/Fluorescence microscopy (Olympus IX 71). Whole blood was drawn from healthy donors into K₂-EDTA-coated vacutainer tubes. Platelet-rich plasma (PRP) was obtained following centrifugation at 200 g for 20 min. The platelets were separated by centrifuging the PRP at 500 g for 15 min, and dispersing the platelet pellet in Tyrode buffer with pH of 7.4 (1 g glucose, 8 g NaCl, 1 g NaHCO₃, 0.2 g CaCl₂, 0.1 g MgCl₂.6H₂O, 0.2 g KCl, 0.05 g NaH₂PO₄.H₂O). 100 μL of washed platelets (2×10⁷ platelets/mL) was added on top of CellNFs dried onto glass coverslips and compared with non-coated coverslips as controls. After incubation for 1 hour at 37° C., coverslips were fixed with paraformaldehyde (3.7%), permeabilized with Triton X-100 (0.1%) and stained with Alexa-488 Phalloidin (Life Technologies, Paisley, UK).

### Hemolysis Study

To study the haemolytic properties of CellNFs in comparison to ORC, direct and indirect haemolysis assays were undertaken. In both methods, red blood cells were isolated from EDTA-anticoagulated blood by centrifugation at 500 g for 5 min and removing the plasma component. In the direct method, the plasma volume was replaced with PBS and freeze-dried CellNFs or ORC were added at 1 and 2 wt % concentrations. In the indirect hemolysis assay, CellNFs or ORC suspended in PBS at concentrations of 1, 2 and 3 wt % were incubated at 37° C. and 5% CO₂ for 24 hours, centrifuged at 1000 g for 20 min, the supernatant was collected and used to replace the plasma volume in the red-blood-cell sample.

The samples for both the direct and indirect methods were then incubated for 3 hours at 37° C., centrifuged at 500 g for 10 min and 100 μL of the supernatant was transferred to a 96-well plate. The optical absorbance of the supernatants was measured at 540 nm using a microplate reader (TECAN Infinite M200 Pro). Haemolysis (H) was calculated using the equation

$$H ( \% ) = ( \frac{Dt - Dnc}{Dpc - Dnc} ) \times 100$$

Where Dt is the absorbance of the test sample, and Dnc and Dpc are the absorbance of negative control (PBS solution) and positive control (deionized water), respectively. The morphology of red blood cells in contact with dried samples were observed under FESEM after processing as described above.

### Shelf Life

The activity of a CellNFs preparation was studied after storage within the lab environment (20° C.) for 1 month.

### Preparation in Different Forms and Sterilization

The samples were prepared into two different forms: gel and freeze dried.

Gel samples were prepared by vacuum filtration of milled samples and adding the appropriate amount of DI water to reach 1 wt. %. The gel can be applied using an applicator. The dry (sponge) samples were prepared by freeze-drying of 1 wt. % gels for 3 to 5 days.

The gel sample was sterilised using an autoclave and the freeze-dried sample was UV sterilized.

The sterilised samples were then checked for procoagulant activity using NATEM. It was found that the sterilisation did not affect the procoagulant activity.

After sterilisation the samples were added to FBS and incubated for 2 weeks in an incubator. No sign of bacteria and fungus growth was observed.

### Statistical Analysis

For each experimental condition of thromboelastometry, an average value was obtained from 3-9 replicates with blood from at least three different donors. The average numbers and standard error (SE) of means were calculated. Statistical significance was determined by performing ANOVA using OriginPro 2015.

### Example 2—Physiochemical Characterization of CellNFs Produced by Ball-Milling

FTIR analysis was performed on un-milled and ball-milled cellulose samples as well as the commercialized ORC product. The main characteristic peaks of cellulose at 3410, 2900, and 1640 cm⁻¹ were observed in the un-milled and milled cellulose samples. These peaks are ascribed to the hydrogen bond stretching vibration of —OH groups, the stretching vibration of C—H, and the bending vibration of absorbed H₂O, respectively. The presence of these peaks in the ball-milled sample indicates that ball-milling did not alter the chemical structure of cellulose. The FTIR spectra of the ORC showed the same 3 peaks but demonstrated an additional peak at 1735 cm⁻¹ that represents carboxylic acid groups associated with oxidation.

XRD patterns of un-milled and milled cellulose samples, along with the commercialized ORC product were obtained. The peaks located around 15-16° and 22° are the main characteristic peaks of the cellulose-Iα crystal structure with preferred orientation along the fibre axis. The result shows that the original un-milled cellulose structure was largely preserved during the milling process, however, as the milling time increased, the Segal's crystallinity (Dcr) decreased from 71% to 64%. Prolonged milling resulted, therefore, in defibrillation of fibres along the crystalline order and, consequently, an increase in amorphous cellulose nanofibres. In addition, a small peak around 30° appeared in the XRD pattern of cellulose samples milled for longer than 45 min. This peak represents zirconia contaminants from the milling process. The amount of zirconia contamination ranged from 0 to 3 vol. % for the milling times from 0 to 180 min. This level of zirconia contamination is blood-compatible and has no thrombogenic behavior. The XRD pattern of the ORC sample showed lower crystallinity.

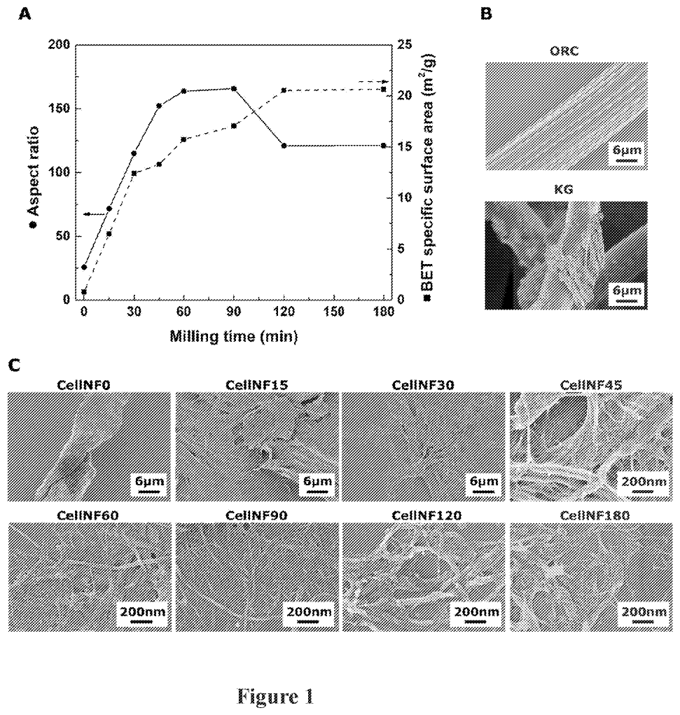

Changes to the specific surface area and aspect ratio of the cellulose through milling was studied (FIG. 1A; FIG. 2; Table 1). A higher aspect ratio results in a higher sedimentation value hₛ/hᵢ and therefore a lower gel point (which correlates with the degree of interconnectivity between fibres in the meshed network of fibres). This is due to fibres with a higher aspect ratio tending to become entangled thus forming a more stable gel in water. Ball-milling induces fibre defibrillation with a reduction in fibre diameter and, in turn, an increase in aspect ratio and specific surface area. Extended ball-milling, however, can result in the shortening of fibres, a decrease in aspect ratio and an increase in specific surface area and gel point.

Ball-milling of cellulose for up to 90 min resulted in a larger specific surface area (17.06±0.11 m²/g), a high aspect ratio (>160) and a low gel point (<1.2 g/L) (FIG. 1A; FIG. 2; Table 1). SEM images of the samples milled for up to 90 min (FIG. 1C) also show that milling caused defibrillation of the fibres with the diameter reduced from 10 μm to less than 100 nm. When the samples were milled for longer than 90 min, their specific surface areas slightly increased (20.53±0.09 m²/g and 20.67±0.09 m²/g) but the aspect ratio decreased and the gel point increased (FIG. 1A; FIG. 2; Table 1). This is likely the result of fibre shortening during the longer milling time. SEM images (FIG. 1C) demonstrate the presence of shorter fibres. Therefore, while the specific surface area of fibres continuously increased with milling time, the aspect ratio of the milled fibres reached a maximum at approximately 90 min prior to decreasing.

TABLE 1
Properties of cellulose fibres after ball-milling

| | | | | Specific |
|---|---|---|---|---|
| Milling Time | Degree of | | | surface area |
| (min) | crystallinity (%) | Gel point | Aspect ratio | (m²/g) |
| 0 | 71 | 48.0 | 25.8 | 0.79 |
| 015 | 69 | 6.27 | 71.5 | 6.49 |
| 030 | 68 | 2.43 | 115 | 12.4 |
| 045 | 68 | 1.38 | 152 | 13.3 |
| 060 | 67 | 1.20 | 164 | 15.7 |
| 090 | 67 | 1.17 | 166 | 17.1 |
| 0120 | 66 | 2.19 | 121 | 20.5 |
| 0180 | 65 | 2.18 | 121 | 20.7 |

In comparison, the ORC sample has a diameter and average aspect ratio similar to those of the native cellulose fibres, as can be seen in SEM images (FIG. 1B). The surface of the ORC sample is completely smooth and consistent throughout the fibre as a result of the molding of the oxidized cellulose pulp. The SEM images of the KG demonstrates the kaolin particles on the surface of the macron size polystyrene and rayon fibres from which the gauze is constituted (FIG. 1B).

### Example 3—Haemostatic Characterization of CellNFs

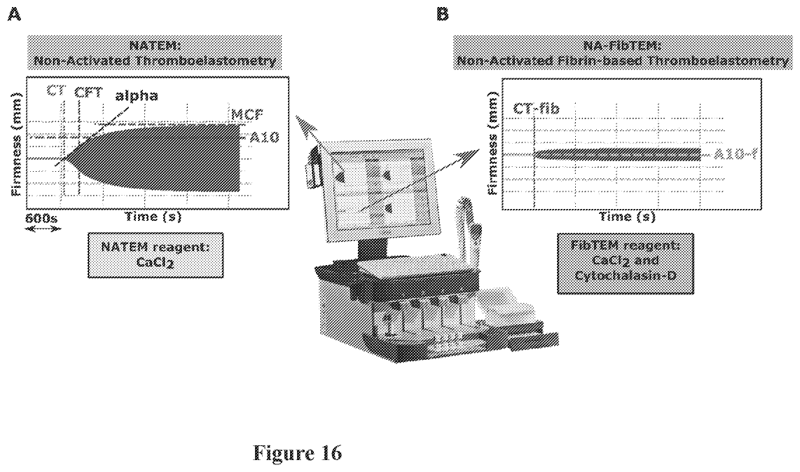

The hemostatic behavior of the CellNFs preparations in whole blood was determined using the ROTEM non-activated assay, NATEM, and the results are tabulated in Table 2. The main parameters in the TEMogram obtained from the ROTEM assay are depicted in FIG. 16. Clotting time, CT, is defined as the time taken for the clot to reach an amplitude of 2 mm and represents the activity of the plasma-coagulation factors. Clot formation time, CFT, is the time taken from the start of coagulation (2 mm), to the time when the clot reaches an amplitude of 20 mm, this phase representing the activation of platelets by thrombin and their interactions with fibrin polymers. The α-angle provides an indication of the kinetics of the CFT. The amplitude of the clot 10 min after the end of the CT is represented by the A10 value. The maximum clot firmness, MCF, is indicative of the strength of the clot, hence the interactions between fibrin and platelets and represents the maximum amplitude of the clot achieved throughout the assay.

The percentage difference of the thromboelastometry parameters of the CellNFs, ORC and KG preparations to those of the internal controls are shown in FIG. 3A and Table 2. Un-milled cellulose powder shortened CT by 25% (±SE 4%). As the milling time increased to 90 min, CT further reduced, gradually to 68% (±SE 2%). The same trend was seen in the CFT with a reduction of 57% (±SE 3%) with 90 min of milling MCF increased from 2% (±SE 1%) at 0 min milling to 10% (±SE 2%) at 90 min milling In all parameters measured, additional milling time beyond 90 min resulted in a reversal of the trend in pro-coagulant properties as a function of milling time.

TABLE 2
Clotting time and maximum clot firmness of ball-
milled cellulose fibres and commercial comparators.

| | CT | MCF |
|---|---|---|
| Haemostat Sample | (% control) | (% control) |
| Suspensions of Haemostats | | |
| (1 wt. % suspension in PBS) | | |
| Ball-milled CellNF0 min | −26 ± 4 | 3 ± 1 |
| Ball-milled CellNF15 min | −53 ± 3 | 4 ± 1 |
| Ball-milled CellNF30 min | −58 ± 3 | 6 ± 1 |
| Ball-milled CellNF45 min | −60 ± 6 | 8 ± 3 |
| Ball-milled CellNF60 min | −64 ± 2 | 9 ± 2 |
| Ball-milled CellNF90 min | −68 ± 2 | 10 ± 2 |
| Ball-milled CellNF120 min | −60 ± 4 | 2 ± 1 |
| Ball-milled CellNF180 min | −62 ± 3 | 7 ± 2 |
| Oxidised Regenerated Cellulose (ORC) | −10 ± 2 | 2 ± 2 |
| Dry Preparations of Haemostats | | |
| (1.5 mg dry form) | | |
| Ball-milled CellNF90 min | −88 ± 6 | 20 ± 4 |
| ORC | −23 ± 2 | 6 ± 1 |
| Kaolin-impregnated gauze (KG) | −73 ± 2 | 2 ± 1 |

In contrast to the ball-milled CellNFs, the ORC suspension resulted in a shortening of CT of 10% (±SE 2.4%), a reduction of CFT by 10% (±SE 3%), an increase in the a angle of 6% (±SE 2.2%) and no significant increase in the MCF.

To summarize, these results revealed that CellNFs ball-milled for 90 min and at a concentration of 1 wt % in PBS dramatically and significantly enhanced hemostasis with the clot occurring more rapidly and the final clot being significantly stronger when compared to the ORC and un-milled cellulose. Further milling reduced these pro-coagulant properties slightly, therefore, 90 min (using the particular mill, balls, speeds, and cellulose slurry concentrations employed) was considered the optimum milling time in terms of haemostatic efficacy. This trend in haemostatic efficacy as a function of milling time correlates with the changes seen in aspect ratio of the CellNFs (FIG. 1A) thus suggesting that the fibre morphology (aspect ratio) has a direct impact on the haemostatic behavior of CellNFs.

The changes in procoagulant parameters, with addition of different concentrations of CellNF90 in PBS, were gradually improved up to 1 wt. % CellNF90 in PBS, and further addition of CellNF90 did not change haemostatic behavior (FIG. 3B).

As CellNFs can potentially be used in the form of a dry sponge (FIG. 15), and ORC and KG are also applied as dry preparations, the procoagulant properties of dry CellNF90 sponge (1.5 mg) were compared with equivalent weights of dry ORC and KG. The reduction in CT was enhanced by all three products (Table 2). This is not unexpected, as, in the dry form, the water adsorption mechanism will act in concert with the procoagulant properties. However, the magnitude of the CT reduction was different between the samples. CellNF90 sponge significantly (p<0.05) reduced clotting time by 88% (±SE 5%) and increased the firmness of the clot by 20% (±SE 4; FIG. 3C). The most common non-absorbable commercial hemostat used in combat, KG, significantly reduced clotting time by 73% (±SE 2%), but increased the clot firmness by just 2% (±SE 1%). ORC, the most commonly-used absorbable hemostat in surgical settings, reduced clotting time by only 23% (±SE 2%) and increased the clot firmness by just 6% (±SE 1%) (FIG. 3C).

The clot microstructures obtained from the NATEM study of control, CellNF90, ORC and KG were studied using SEM with 5×10³ x magnification (FIG. 3D). In the clot of the control PBS sample, blood components are interwoven with fibrin fibres (FIG. 3D). With the addition of CellNF90 to the blood, a mesh-like structure formed by the CellNF90 intermingled with blood components and fibrin fibres were prominent (FIG. 3D). The KG sample also showed an obvious fibrin network as a result of activation of Factor XII of the intrinsic coagulation cascade by kaolin. In comparison, the clot from the ORC sample revealed red blood cells adsorbed to and deformed by the oxidized fibres but there was a distinct absence of fibrin fibres. The results of this SEM study support the notion that nanoscale cellulose fibres with a large aspect ratio and specific surface area are more effective at promoting the formation of fibrin resulting in enhanced hemostasis.

In order to further clarify the mechanism through which CellNF90 interacts with the natural haemostatic system, non-activated FibTEM assays (NAFibTEM), in which platelet activity is inhibited by the addition of cytochalasin-D thus excluding their contribution to clot formation, were performed alongside NATEM assays on the CellNF0, CellNF90, CellNF180 and the commercial ORC sample in the suspension form with concentration of 1 wt. %. A significant reduction in clot amplitude was observed in the NAFibTEM (FIG. 9) compared with that in the NATEM (FIG. 9) emphasising the contribution of platelets to the clot that was inhibited by cytochalasin D in this assay.

The relative values of CT and A10 obtained from the NATEM assays (whole blood with platelet activity) and the NAFibTEM assays (whole blood without platelet activity) in the presence or absence of the hemostatic materials are shown in FIG. 4A. In addition, the effect of the materials on the platelet contribution to the clot can be calculated by subtracting the NAFibTEM A10 value from the NATEM A10 value (FIG. 4A and Table 3).

TABLE 3
Plasma contribution and platelet contribution to clotting in the
presence of cellulose fibre meshes (1 wt. % suspended in PBS).

| | Plasma contribution | Platelet contribution |
|---|---|---|
| Preparation | (%) | (%) |
| CellNF0 | 0 ± 0 | 6 ± 6 |
| CellNF90 | 28 ± 10 | 17 ± 3 |
| CellNF180 | 30 ± 6 | 5 ± 5 |
| ORC | 4 ± 3 | 4 ± 1 |

For CellNF0, the shortening of CT in the NATEM assay was 27% (±SE 2%) but no significant effect was observed in the NAFibTEM assay. CellNF0 did not significantly increase A10 in both the NATEM and NAFibTEM assays. This result implies that the minimal haemostatic activity of macro-scale cellulose fibres relies on the presence of both plasma components and platelets.

In contrast, CellNF90 shortened CT in the presence and absence of platelet activity by 68% (±SE 3%) and 65% (±SE 2%) respectively. Additionally, CellNF90 increased the contribution of platelets and plasma to the A10 by 17% (±SE 3%) and 28% (±SE 10%) respectively. Thus, the mechanism of enhanced haemostasis by CellNF90 is associated with the ability to activate coagulation promoting thrombin formation which induces fibrin formation and enhances platelet activation.

In the results obtained with CellNF180, clotting time decreased by 62% (±SE 3%) and 66% (±SE 2%), respectively in whole blood and platelet-inhibited blood. In comparison to CellNF90, CellNF180 showed a similar enhancement of plasma contribution, 30% (±SE 6%), but a lower level of platelet activation (5% ±SE 5%).

The trend in platelet activity and plasma contribution as a function of milling time is in good correlation with the change in aspect ratio and specific surface areas, respectively, as a function of milling time (FIGS. 1A and 4A). The positive domain of fibrinogen has previously been shown to irreversibly adhere to a negatively charged surface. Thus, it is proposed that the higher specific surface area of CellNF90 leads to greater adsorption of nano-sized plasma proteins such as fibrinogen (475 Å) and induces faster fibrin formation. In contrast, ORC prevents the formation of fibrin due to its acidic nature. We speculate that the enhanced fibrin formation of CellNF90 is a result of increased thrombin production following the induction of plasma coagulation. The production of thrombin also results in the activation of platelets thus enabling the platelet receptor, αIIbβ3, to bind to the fibrin strands.

In comparison to the CellNFs preparations, the results obtained with ORC in the NATEM and NAFibTEM assays shows negligible enhancement of platelet activation or plasma coagulation (FIG. 4A) suggesting that the hemostatic properties of ORC are principally due to fluid absorption.

To confirm that the ROTEM results obtained with CellNF90 are due to the promotion of a coagulation cascade and not just due to physical impedance of the pin rotation, we performed the NATEM assays with CellNF90 but without the addition of CaCl₂ necessary for enzymic activity in both platelets and plasma coagulation. As shown in FIG. 4B, without Ca²⁺ ions no resistance to the pin rotation was measured thus confirming that CellNF90 enhances haemostasis through enzymatic reactions of the coagulation cascade.

As CellNF90 was demonstrated to enhance plasma coagulation, studies were undertaken to investigate the factors involved in this process using heparinized blood and Factor IX, XI, and XII deficient plasma.

Factors IX, XI, and XII are involved in the intrinsic pathway (contact activation pathway) of plasma coagulation. Using Factor-XI and -XII deficient plasmas, CellNF90 could not induce hemostasis (FIG. 4C). Since Factor IX could be activated by thrombin released from other interactions in the coagulation cascade (through Factor VII and X), the clotting was not fully inhibited by using Factor-IX deficient plasma. However, there was no difference between CellNF90 and control with contact-dependent activation from CellNF90 (FIG. 4C). These results confirm that the main mechanism in which CellNF90 enhances clot formation is through the intrinsic pathway. As the density of negative charge on biomaterials surfaces play a significant role in their thrombogenic behavior through intrinsic pathway, a higher number of negatively charged sites in the CellNF90 with higher specific surface areas promoted the reactivity of coagulation factors resulting in thrombin generation and enhanced fibrin formation.

Following the addition of a titration of heparin to the blood, the clotting time increased from about 600 s to about 2500 s (FIG. 4D; Table 4). Heparin impedes the common pathway of coagulation by deactivating Factor X and thrombin. The addition of CellNF90 to heparinized blood reduced clotting time by 54% (±SE 2%), thus slightly less effective in comparison with the effect of CellNF90 on non-heparinized blood (68% (±SE 1%). The results indicate that the hemostatic action of ball-milled CellNFs solely relies on the intrinsic pathway. Thus, CellNFs with a high surface area and a high aspect ratio are capable of inducing significant haemostasis in heparinised blood.

TABLE 4
Clotting time reductions induced by CellNF90 (1 wt. % suspension
in PBS) in whole blood in the presence of increasing heparin.

| Heparin dose | Clotting time (s) of | % reduction in clotting time |
|---|---|---|
| (μg/mL blood) | heparinized blood | induced by CellNF90 |
| 0 | 631 ± 17 | −68 ± 1 |
| 0.0625 | 1078 ± 108 | −58 ± 1 |
| 0.125 | 1612 ± 381 | −50 ± 1 |
| 0.25 | 2503 ± 380 | −52 ± 3 |
| 0.5 | ND | ND |

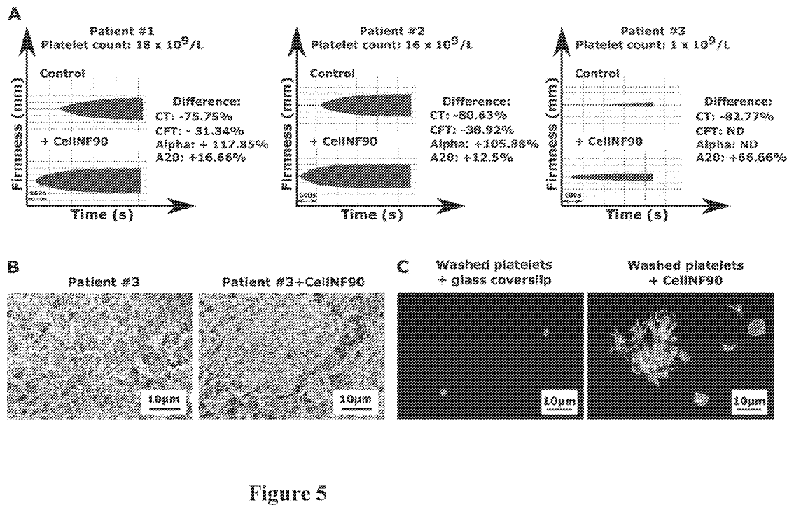

Thrombocytopenic patients are at an increased risk of spontaneous bleeding when platelet counts are less than 20×10⁹/L of blood. The platelet counts in healthy individuals range from 150 to 400×10⁹/L. In order to control bleeding, thrombocytopenic patients are normally given platelet transfusions. As can be seen in FIG. 5A, the addition of CellNFs to the blood of patients with 18, 16 and 1×10⁹ platelets/L shortened clotting time from 75 to 82%, confirming that the main hemostatic mechanism of CellNFs is the promotion of fibrin formation. FIG. 5B shows the SEM images of the clot obtained from the patient #3 (with platelets count of 1×10⁹ per L of blood) with and without addition of CellNF90. While plasma coagulation and subsequent fibrin formation was impaired due to low platelet numbers, the presence of CellNF90 assisted fibrin network formation (FIG. 5B, Table 5).

TABLE 5
Reductions in clotting time induced by CellNF90 (1 wt.
% in PBS) in the presence of varying platelet counts.

| Platelet numbers in | | |
|---|---|---|
| patients' blood | Clotting time | % CT reduction with |
| (×10⁹ per L) | (NATEM) (s) | CellNF90 |
| 18 | 928 | −75.5 |
| 16 | 754 | −80.6 |
| 1 | 1469 | −82.7 |

To determine the activation state of platelets on CellNF90, the actin structures of platelets on CellNF90 were stained and imaged via fluorescence microscopy (FIG. 5C). Platelets were trapped within the mesh-like structure of CellNF90 and were observed to have formed filopodia. In comparison, few platelets were captured on the untreated glass slide and platelets remained inactive.

### Example 4—Lysis of Blood Components Study

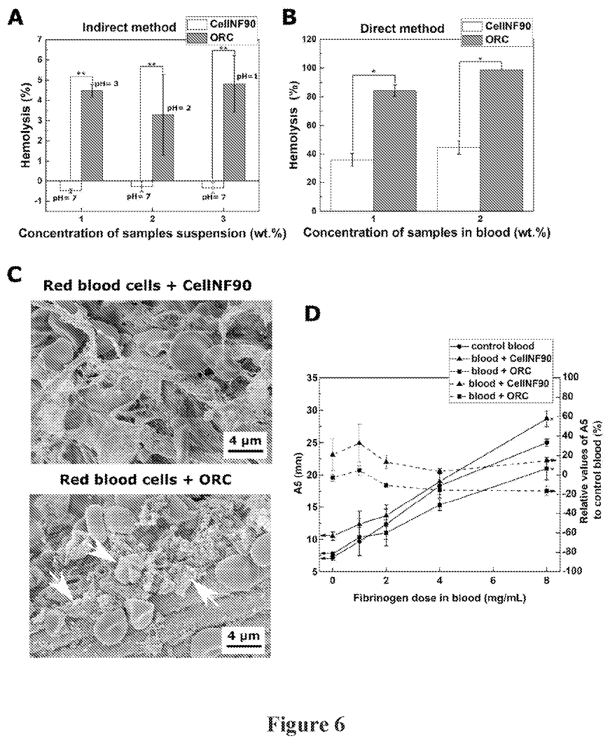

Investigations into the lysis of red blood cells (haemolysis) following contact with ORC and CellNF90 shows that the supernatant of CellNF90 after 24 hours of incubation had a pH of 7 and did not induce haemolysis, regardless of the supernatant concentrations. In contrast, the pH of the supernatant of ORC decreased from 4 to 1 when the concentration of ORC was increased from 1 to 3 wt % as a result of H⁺ ions released from ORC. The low pH environment of ORC caused up to 5% (±SE 1%) haemolysis (FIG. 6A; Table 6).

In a direct contact experiment, the addition of dried CellNF90 to red blood cells at concentrations of 1 and 2 wt %, caused 36% (±SE 5%) and 44% (±SE 5%) haemolysis, respectively. In contrast, the addition of ORC at concentrations of 1 and 2 wt % caused 84% (±SE 4%) and 99% (±SE 5%) haemolysis, respectively (FIG. 6B; Table 6). SEM images demonstrate that red blood cells in contact with CellNF90 preserved their normal morphology, while many of red blood cells in contact with ORC were wrinkled and hollowed (FIG. 6C). For both CellNF90 and ORC samples, the direct method caused significantly higher haemolysis than the indirect method (Table 6). This may be attributed to the mechanical forces applied to red blood cells by centrifugation in contact with dried samples.

TABLE 6
Haemolysis (vs control) of red blood
cells caused by cellulose fibres

| | Concentration | Haemolysis |
|---|---|---|
| Cellulose fibre | (wt. %) | (% of controls) |
| Indirect Assay | | |
| CellNF90 | 1 | −0.5 ± 0.1 |
| CellNF90 | 2 | −0.3 ± 0.3 |
| CellNF90 | 3 | −0.3 ± 0.3 |
| ORC | 1 | 4.5 ± 0.3 |
| ORC | 2 | 3.3 ± 2 |
| ORC | 3 | 4.8 ± 1 |
| Direct Assay | | |
| CellNF90 | 1 | 36 ± 4 |
| CellNF90 | 2 | 44 ± 0.2 |
| ORC | 1 | 84 ± 4 |
| ORC | 2 | 98 ± 0 |

In the SEM image of the clot of ORC (FIG. 3D), fibrin fibres were not observable, hence the possibility of the acidic nature of ORC preventing fibrin formation was studied (FIG. 6D). With the addition of fibrinogen to blood, the amplitude of the clot 5 min after the onset of clotting (A5) increased from 7 to 25 mm (FIG. 6D; Table 7). The addition of CellNFs to the fibrinogen-supplemented bloods enhanced the A5 from 10 to 28 mm over the fibrinogen concentration range equating to a maximum 17%±5% increase. In comparison, the addition of ORC to the fibrinogen-supplemented blood reduced the A5 from 7 to 21 mm over the fibrinogen concentration range equating to a maximum decrease of 8%±4%.

Thus, ORC reduced the blood pH causing haemolysis and inhibiting fibrin formation thus preventing maximal coagulation. In comparison CellNFs promoted coagulation by enhancing fibrin formation and leaving red blood cells intact.

TABLE 7
Changes in clot amplitude at 5 min (A5) induced
by CellNF90 and ORC (1wt. % in PBS) in whole
donor blood supplemented with fibrinogen.

| Fibrinogen dose | Control | CellNF90 | ORC |
|---|---|---|---|
| (mg/mL blood) | A5 values | (% change in A5) | (% change in A5) |
| 0 | 7 ± 0.5 | +21 ± 16 | −2.6 ± 3 |
| 1 | 10 ± 2 | +33 ± 19 | +4.4 ± 4 |
| 2 | 12 ± 2 | +13 ± 7 | −11 ± 0.6 |
| 4 | 18 ± 2 | +3.6 ± 2 | −16 ± 5 |
| 8 | 25 ± 0.6 | +15 ± 3 | −16 ± 5 |

### Example 5—In vivo haemostatic studies

To design a successful hemorrhage model using mice, all the physiological variables (mice strain, age, and gender) were optimized to obtain severe bleeding condition. Thus, 8 weeks old male Balb/c mice (ASD867) were used for this experiment.

Mice were weighed then anaesthetized by intraperitoneal (I.P.) injection of the anesthetic cocktail (50 mg/Kg Ketamine and 10 mg/Kg Xylazine), and the anesthetized state was maintained using isoflurane administration. The mice were then placed in a tissue lined cage under an appropriately distanced heat lamp to keep the mice warm until unconscious. Once unconscious and pedal reflexes were confirmed absent, the mouse was placed in a supine position on a heated surgical area (37° C.). All four limbs of the mouse were secured with tape, and the abdomen was shaved with a razor. Betadine-soaked and ethanol-soaked cotton buds were applied to the abdomen to sterilize the surgery site. A ventral midline laparotomy incision was made starting at the xiphoid process and extending caudally to allow complete exposure of the liver. Then, two pre-weighed non-adherent absorption triangles (made of double sided low adherent pad: Sterile Aeropad®, Aero Healthcare, NSW, Australia) were inserted in the abdomen against the right and left abdominal wall. The absorption triangles were kept clear of the liver to avoid a packing hemostatic effect following liver laceration. More pads were used when the pads were soaked in blood. The left-middle lobe of the liver was carefully elevated and ⅔ of the lobe was removed with surgical Iris scissors. The excised liver was stored and weighed in a pre-weight Eppendorf containing 500 μL PBS to assure the consistency. The animals were pre-assigned to 3 treatment groups (control (nothing), ORC, and sponge-form CellNFs) by a person other than the operator who was blinded to this assignment at the time of liver excision. Then, 3 pieces of 5 mg samples were place on the liver injury. The experiment was terminated 15 minutes post liver injury at which time the absorption triangles were collected and, later, weighed to calculate the blood loss. Finally, the mice were euthanized via cervical dislocation prior to cessation of isoflurane. The liver was collected, and was first fixed with 2.5% glutaraldehyde in a PBS buffer for 24 hours at room temperature, then washed with PBS, deionized water and ethanol, subsequently dried using a super critical dryer (40° C., 90 bar; Balzers CPD 030). The dried samples were sputter-coated with platinum prior to FESEM imaging.

10 mice were assigned to each experimental group to reduce the effects of experimental and physiological variables. The average value of blood loss was obtained. The mean and the standard error (SE) of the mean were calculated using OriginPro 2015. Statistical significance was determined by performing ANOVA also using OriginPro 2015.

The excised livers were between 200 to 300 mg without any significant difference among experimental groups, and all 30 mice survived the 15 min procedure time. As can be seen in FIG. 7A, without applying any hemostatic agent on the wound, the bleeding ceased due to the body's natural coagulation process, and the total blood loss was 549 mg (±SE 44 mg). Applying CellNFs (expressed as “CNFs” in FIG. 7) reduced the blood loss significantly by 38% (±SE 10%) to 345 mg (±SE 30 mg). Applying Surgicel® (ORC) reduced the blood loss by 15% (±SE 10%) to 465 mg (±SE 36 mg), and the difference between control and ORC was not statistically significant. The photos and the FESEM images of the remaining liver lobe portion showing the formation of the clot in contact with the samples (FIG. 7B), illustrates that ORC dehydrated and blackened the liver tissue consistent with the hemostatic action being to absorb fluid and the acidity of the product resulting in oxidation of hemoglobin. The CellNFs with neutral pH maintained the normal liver tissue structure. These results correlate well with in vitro thromboelastometry findings, showing that CellNFs outperforms ORC in arresting the blood loss with enhancing the normal blood coagulation process.

### Example 6—In Vitro Biocompatibility Studies

Further in vitro biocompatibility studies other than those described earlier (“Hemolysis study”). These included evaluation of proliferation rate of endothelial cells and fibroblast cells, and extended incubation periods.

In order to evaluate the proliferation rate of the endothelial cells and fibroblast cells in contact with CellNFs sample in comparison with ORC and KG, the MTT (3-[4,5-dimethylthiazol-2-yl]-2,5-iphenyltetrazoliumbromide) assay were conducted. In this regard, the sterilized samples were first suspended in PBS at concentrations of 1, 2 and 3 wt. % and incubated at 37° C. under a 5%-CO₂ atmosphere for 24 hours. After centrifugation at 1000 g for 20 minutes, the supernatants were collected.

Human dermal microvascular endothelial cells (HMEC-1) were cultured in the prepared endothelial cell culture medium, and the cells (passage numbers of 12, 13, and 14) with density of (5×10⁴ cell/well) were seeded in 100 μL of culture medium into wells of 96 well-plate (NUNC™, Thermo Fisher Scientific, Waltham MA). Human fibroblast cells (HMEC-1) were also cultured in the prepared the cell culture, and the cells (passage numbers of 3, 4, and 5) with density of (5×10⁴ cell/well) were seeded in 100 μL of culture medium into wells of 96 well-plate (NUNC™, Thermo Fisher Scientific, Waltham MA).

After incubation at 37° C. under a 5%-CO₂ atmosphere for 24 hours, the media was changed and replaced with 100 μL of fresh culture medium and samples' supernatants. The plates were kept in the incubator for 3, 24 and 48 hours. Then, the culture medium was removed from each well and replaced with 100 μL of serum free media and 10 μL of MTT reagent (0.5 mg/mL) solution. After 4 hours of incubation, 100 μL isopropanol containing 0.04 nM of hydrogen chloride was added to each well and mixed thoroughly by repeated pipetting to dissolve the generate purple crystals. Then the optical density (OD) of formazan in the solution was measured at 570 nm with the reference wavelength of 630 nm using a (TECAN Infinite M200 Pro). The cell proliferation were calculated using the following equation:

$$cell proliferation ( \% ) = \frac{S - {C}_{sn}}{{C}_{p} - {C}_{n}} \times 100$$

where S is absorbance of samples in contact with cells. Cₛₙ is the absorbance obtained from negative control of each sample containing samples' supernatant added to the media without cells. Cₚ and Cₙ were the absorbances related to the positive (PBS added to the cells) and negative control (PBS added to the media without cells), respectively. To obtain statistically valid calculations, each condition of samples was repeated 9 times using three different passages of the cells. The mean, the standard error (SE) of the mean and the statistical significance was determined using OriginPro 2015.

The results are displayed in FIGS. 8A and 8B. CellNFs (expressed as “CNFs” in FIG. 8) and KG had no significant effect on proliferation of cells in comparison with control. However ORC, by making culture media acidic, exhibit significant toxicity towards endothelial cells and the cell proliferation reduced significantly with the increase in concentration and incubation time. The same trend was also observed in contact with fibroblast cells, however, the fibroblast cells seem to be less vulnerable to acidic environment comparing to endothelial cells. These results demonstrated the compatibility of non-oxidized CellNFs as topical hemostatic agents with minimum side effects on bystander cells.

### Example 7—In Vivo Biocompatibility Studies

Mice were weighed then anaesthetized by I.P. injection of the anesthetic cocktail (50 mg/Kg Ketamine and 10 mg/Kg Xylazine), and the anesthetized state was maintained using isoflurane administration. Then, the mice were placed in a tissue lined cage under an appropriately distanced heat lamp to keep the mice warm until unconscious. Once unconscious and pedal reflexes were confirmed absent, the mice were placed in a ventral position with sterile ophthalmic gel (Poly Visc, Alcon, Geneva, Switzerland) applied and the feet secured with gauze and tape. The mice were shaved with an electric clipper to remove hair from the skin on the upper back. Betadine-soaked and ethanol-soaked cotton buds were applied to the surgical site. A 5 mm subcutaneous incision along the neck region was made using a sterile carbon steel surgical blade (size 22, code 0208, Swann Morton, Sheffield, England). Subcutaneous pockets were made on each side of the incision using a blunt surgical instrument. The materials pre-prepared in cone shape (0.05 cm³), were inserted into each pockets using forceps. The animals were divided into 3 treatment groups, control, ORC and CellNFs (sponge form). The wound was sutured using synthetic monofilament polypropylene sutures with stainless steel circle reverse cutting needle (Cat # MLPP-PPL408015SL, Filaprop, Meril, Gujarat, India), and analgesia (Buprenorphine 0.05 mg/Kg) was injected subcutaneously. The toe nails were cut short to reduce the ability of mice to scratch the wound. Then, the mice were removed from the isoflurane nose cone, placed in a cage under a heat lamp until recovered, then placed into a separate cage with environmental enrichment (tissues papers and toilet roll). Each treatment group was further divided into 4 sub-groups according to the time course of the experiment (3 days, 1 week, 2 weeks, and 4 weeks). 3 animals were assigned to each subgroup to validate the reliability of the experiment conditions. Food, water and cage care were undertaken according to the Australian Phenomics Facility protocol. Animals were observed and monitored hourly for a 4-hour period, then twice daily for three days, then once daily for the remainder of the experimental period. The wounds were photographed on weekly basis. At the end of the assessment stage, the animals were euthanized using CO₂ gas. Subsequently, the dorsal skin and subcutaneous tissue were excised and fixed by 10% formalin. After gradient ethanol dehydration, the samples were embedded in paraffin, sectioned and stained with hematoxylin and eosin (HE). The histopathological investigations were performed by a pathologist blinded to the samples. The presence of foreign material, foreign body reaction, inflammation (acute and chronic), edema, fibrin, granulation tissue and scarring were scored.

The in vivo model was employed to assess the biocompatibility and biodegradation of the CellNFs in comparison with ORC, as a degradable agent, over time in the mice body. In this model, the samples with the same size (FIG. 9) were implanted subcutaneously in the back of the mice. Our initial assessments after surgery showed that all mice recovered after 30-50 min and normal behavior, grimace score of (0), and no weight loss was observed for up to 4 weeks. No redness or inflammation was observed in the mice assigned to the control procedure and the CellNFs (expressed as “CNFs” in FIG. 9), and the sutures fell out in the second week as a result of complete wound healing. The surgical site of the mice implanted with the commercial agent ORC, in comparison, was reddened and swollen for up to three days post-surgery. At the termination point, CellNFs was detectable for up to 4 weeks, while the degradable commercial agent, ORC, was completely degraded 1 week following implantation.

Histological examination of the formalin-fixed, paraffin-embedded, haematoxyloin and eosin-stained tissues excised from the site of implantation was undertaken by an independent senior Pathologist. At 3 days, tissue edema and inflammation was observed for the control, CellNFs and ORC treatment groups, as expected. The animals implanted with CellNFs had a more intensive neutrophil infiltrate than the ORC group. Both the CellNFs and ORC treated groups showed more tissue edema and a denser neutrophil infiltrate than the control group. The amount of fibrin deposition was similar in the CellNFs and ORC treated groups while no fibrin deposition was observed in the control group.

At one week, there was more tissue edema and a denser neutrophil infiltrate in the ORC treated group than the CellNFs treated mice. Both groups had a similarly dense chronic inflammatory cell infiltrate and granulation tissue formation. The foreign body reaction to the topical hemostatic agents was more intense in the CellNFs treated group. In contrast the sections of the skin from the control group showed early scar formation with absence of acute inflammation and tissue edema. Scattered chronic inflammatory cells were seen in the dermis without a foreign body type giant cell reaction.

At two weeks, there was no tissue edema and less granulation tissue formation evident in any of the groups, as expected. The control tissue showed scar formation and scattered chronic inflammatory cells. The mice treated with CellNFs showed a marked chronic inflammatory cell infiltrate with plentiful foreign body type giant cells and early scar formation. The mice treated with ORC showed less chronic inflammation and fewer foreign body type giant cells with early scar formation.

At four weeks, dermal scar tissue was more prominent in the ORC treated groups than the CellNF or control groups where the dermal tissue had largely returned to normal. A foreign body reaction to the topical hemostatic agents was, however, active in the CellNF group but there was a marked reduction in the size of the inflammatory response compared to two weeks in both treatment groups.

### Example 8—Chitin Nanofiber Studies

Chitin powder extracted from shrimp (MDL number: MFCD00466914) was purchased from Sigma-Aldrich (Missouri, United States). Cerium-doped zirconia balls (Zirconox®) with a diameter of 0.4-0.6 mm were purchased from Zirconox® (Jyoti Ceramic, Maharashtra, India). Lab water purification systems (PURELAB flex 3) were used to generate deionized water with a purity of 18.2 MΩ·cm. Absolute ethanol (MDL number: MFCD00003568), sterile-filtered Dulbecco's phosphate-buffered saline (PBS) 1× with the pH of 7.4 without calcium chloride, and magnesium chloride (MDL number: MFCD00131855) were purchased from Sigma-Aldrich (Missouri, United States). Star-TEM reagent containing CaCl₂ (000503-01), Fib-TEM reagent containing CaCl₂ and cytochalasin-D (000503-06), and InTEM reagent containing thromboplastin phospholipid and ellagic acid (000503-02) were obtained from Tem Innovations GmbH (Munich, Germany).

The chitin powder was suspended in deionized water at a concentration of 0.5 wt. %. Cerium-doped zirconia balls were added to the suspension with a ball-to-powder mass ratio of 80:1. The mixtures were milled using a Spex 8000M Mixer/Mill (model 8000M) with a shaking frequency of 950 rpm for 0, 1.5, 3, 4, 5, and 6 hours. The samples were denoted according to the milling time, as CTNF0, CTNF1.5, CTNF3, CTNF4, CTNF5, and CTNF6.

In order to study the chemical structure of the samples, Fourier-transform infrared (FTIR) spectroscopy was performed on freeze-dried samples using a Perkin-Elmer Lambda spectrometer in a range between 500 and 4000 cm⁻¹ at a resolution of 4 cm⁻¹.

The crystal phases of the pressed freeze-dried samples were studied by X-ray diffractometry (XRD) using a Bruker D2 Phaser X-ray diffraction instrument at 30 kV and 10 mA, in a diffraction-angle range from 10° to 60° with a Cu Kα radiation (λ=1.542 Å). The degree of crystallinity, D_{cr}, was estimated using the following equation:

$${D}_{cr} = \frac{{I}_{110} - {I}_{am}}{{I}_{110}} \times 100$$

where I₁₁₀ is the maximum intensity of the peak in the 110 crystal plane and Iₐₘ is the intensity of the amorphous diffraction at approximately 15-16°.

The amount of zirconia contamination introduced into CTNFs by the ball-milling process was measured by comparing the sample weight before and after burning off chitin in air. The heat treatment was carried out on 1 g of freeze-dried samples in air at 1000° C. for 2 hours in a pre-dried aluminum crucible.

The positive surface charge of nanofibers was confirmed by measuring the zeta potential of samples dispersed in deionized water with a concentration of 0.05 wt. %, using a Zetasizer (NanoZS90, Malvern Instruments, UK).

The average aspect ratio (length to diameter) of fibers and specific surface area of freeze-dried samples were measured as described earlier.

All rheological behavior of the gels was studied using a Kinexus rheometer with a cone and plate geometry at 25° C. Oscillatory strain sweep was performed from 0.01 to 100% at a constant 1 Hz frequency.

In these studies a third ROTEM assay mode (as well as NATEM and NAFibTEM) was used: intrinsic pathway thromboelastometry (InTEM) in which CaCl₂, thromboplastin phospholipid and ellagic acid were added prior to measurement to initiate the plasma coagulation through intrinsic (contact activation) pathways.

To analyze which coagulation pathways were induced by CTNF5, NATEM and InTEM assays were performed in plasma that is deficient in Factor-IX, XI, or XII (Instrumentation laboratory, Massachusetts, United States) in the presence and absence of CTNF5 (in suspension form at 1 wt. %). Platelet-poor plasma (PPP) was run concurrently as a control plasma without deficiency. To obtain PPP, whole citrated blood was centrifuged at a centrifugal force of 500 g for 5 minutes. The plasma (without buffer coat) was collected and centrifuged again with a force of 700 g for 10 min, and the PPP without pelleted platelets was collected.

### Results

The FTIR spectra of chitin matches the FTIR spectra of chitin derived from shrimp recorded in the literature. The peak at approximately 3400 cm⁻¹ is attributed to an O—H and N—H stretching band. The peaks at 1659 and 1551 cm⁻¹ correspond to the stretching of the C═O group (amide I) and amide II (N—H bending), respectively. The FTIR spectra showed no change with milling time, indicating that the ball-milling process did not alter the chemical structure of chitin.

The original chitin structure was largely preserved during the milling process. In fact, two diffraction peaks at approximately 9.5° and 19.5° corresponding to the (020) and (110) crystal planes of chitin, respectively, can be seen in all the samples. However, prolonged milling resulted in defibrillation of fibers along the crystalline order and, consequently, a decrease in the degree of crystallinity (D_{cr}) from 84% to 75%. The peak around 30° represents zirconia contaminants from the milling process, ranging from 0 to 26 wt. % for the milling times up to 6 hours. Zirconia is biocompatible and has been used in a variety of biomedical applications. There is no report of its thrombogenice behavior in the literature.

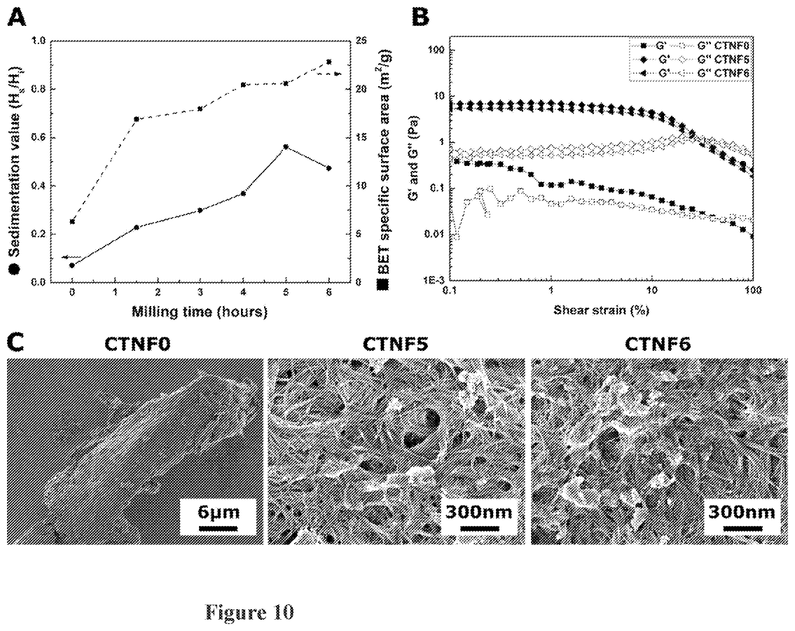

FIG. 10A shows the sedimentation value, representative of aspect ratio, and specific surface area of the chitin as a function of milling time. It is to be noted that a higher aspect ratio results in a higher sedimentation value Hₛ/H₁, due to the fact that fibers with a higher aspect ratio tend to become entangled thus forming a more stable suspension in water.

Ball-milling induces fiber defibrillation with a reduction in fiber diameter and, in turn, an increase in aspect ratio and specific surface area. Extended ball-milling, however, can result in the shortening of fibers, a decrease in aspect ratio and an increase in specific surface area. Ball-milling of chitin for up to 5 hours resulted in a higher specific surface area (20 m²/g) and a high aspect ratio (sedimentation value: 0.6; aspect ratio of about 155) (FIG. 10A). SEM images of the samples milled for up to 5 hours (FIG. 10C) confirmed that milling caused defibrillation of the fibers and a decrease in fiber diameter from 10 μm to less than 100 nm. When the samples were milled for longer than 5 hours (6 hours; CTNF6), their specific surface areas slightly increased (22 m²/g) but the aspect ratio decreased (sedimentation value: 0.47; aspect ratio of about 100; FIG. 10A). This is likely due to the fiber shortening induced during the longer milling

In a strain sweep mode of oscillatory rheology, the viscoelastic behavior of the gels is assessed under an increasing oscillating strain (strain sweep) at a constant frequency. The elastic modulus G′ and viscous modulus G″ describe the solid-like behavior and the liquid-like behavior of the CTNFs gel, respectively. As shown in FIG. 10B, at low shear stresses, the linear viscoelastic region (LVR) represents independence of G′ and G″ from the shear stress. Within this region, the gel is acting solid-like. At a critical shear stress, the gel yields when reaches a “cross-over point”. Past this critical stress, the viscous regime dominates (G″>G′), indicating that the network structure has been formed. CTNF5 and CTNF6 both possess a distinct linear viscoelastic region and a yield point, while CTNF0 does not present a viscoelastic behavior. Since the viscoelastic behavior of nanofibers is significantly dependent of their aspect ratio, the rheological studies supports the optimum aspect ratio of CTNF5.

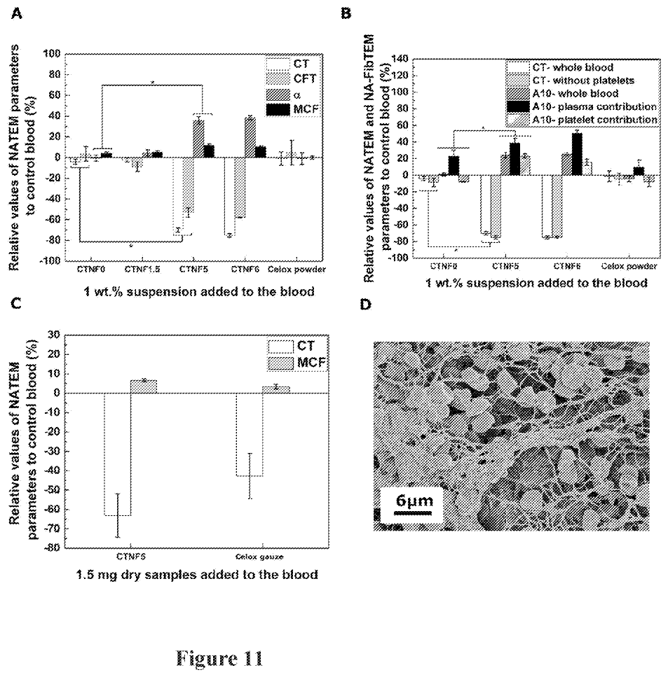

The percentage difference of the thromboelastometry parameters of the CTNF0 (aspect ratio of about 20), CTNF1.5 (aspect ratio of about 60), CTNF5 (aspect ratio of about 155) and CTNF6 (aspect ratio of about 100) in a suspension form as compared to those of the internal controls, are shown in FIG. 11A. CT, the parameter representing the initiation of plasma-coagulation factors activity, was shortened by 4% (±SE 2%) for un-milled chitin suspension. As the milling time increased to 1.5 hours, the reduction in CT was not changed significantly. After milling for 5 hours, the CT further reduced to −70% (±SE 2%). The same trend was seen in CFT, the parameter representative of the platelet activation by thrombin and their interactions with fibrin polymers, with a reduction by 53% (±SE 4%) at 5 hours of milling time. The relative change in a angle, the parameter indicating the kinetics of clot formation, increased to +36% (±SE 4%) with 5 hours of milling MCF, the parameter showing the strength of the final clot, increased from +4% (±SE 1%) at 0 hour, to +12% (±SE 1%) at 5 hours of milling. In all parameters measured, longer milling time than 5 hours did not induce a significant change in the clotting profiles.

Comparing CTNF5 with cellulose nanofibers (CellNF90) obtained by ball-milling for 1.5 hours in our previous work, the initiation of the clot and the strength of the final clot were not significantly different. However, pace of the clot formation, represented with a angle, was slightly faster with CTNF5 (Table 8).

TABLE 8
Sedimentation value, specific surface area, zeta potential, NATEM and NAFibTEM results
of chitin nanofibers (CTNFs) in comparison with cellulose nanofibers (CellNFs).

| | | | | | | | | Relative values of | |
|---|---|---|---|---|---|---|---|---|---|
| | | | | | | | | NAFibTEM parameters to | |
| | | Specific | Zeta | Relative values of | | | | control blood (%) | |
| | Sedimentation | surface | potential | NATEM parameters to | | | | Plasma | Platelet |
| | value | area | in pH 7 | control blood (%) | | | | contribn in | contribn in |
| Sample | (Hₛ/Hᵢ) | (m²/g) | (mV) | CT | CFT | α | MCF | A10 (%) | A10 (%) |
| CellNF0 | 0.05 | 1 | −28 ± 2 | −25 ± 4 | −20 ± 3 | 10 ± 2 | 2 ± 1 | 0 ± 0 | 6 ± 6 |
| CTNF0 | 0.07 | 6 | +26 ± 1 | −4 ± 2 | 3 ± 6 | −0.5 ± 3 | 4 ± 1 | 26 ± 10 | −7 ± 1 |
| CellNF90 | 0.75 | 17 | −28 ± 1 | −68 ± 2 | −43 ± 3 | 27 ± 4 | 10 ± 2 | 28 ± 10 | 17 ± 3 |
| CTNF5 | 0.56 | 21 | +26 ± 1 | −70 ± 2 | −53 ± 4 | 36 ± 4 | 12 ± 1 | 36 ± 8 | 23 ± 2 |
| CellNF180 | 0.61 | 20 | −30 ± 1 | −62 ± 3 | −30 ± 3 | 14 ± 3 | 7 ± 2 | 30 ± 6 | 5 ± 5 |
| CTNF6 | 0.48 | 23 | +27 ± 1 | −75 ± 2 | −57 ± 1 | 38 ± 2 | 10 ± 1 | 50 ± 4 | 15 ± 4 |

The increase in pro-coagulant properties with milling time up to 5 hours, supports the substantial impact of morphology on the hemostatic properties. In order to further clarify this hypothesis, non-activated FibTEM assays (NAFibTEM), in which platelets are inhibited by the addition of cytochalasin-D thus excluding their contribution to clot formation, were performed alongside NATEM assays on the CTNF0 , CTNF5, CTNF6 each suspended at 1 wt. % in PBS.

The relative values of CT and A10 obtained from the NATEM assays (whole blood with platelet activity) and the NAFibTEM assays (whole blood without platelet activity) in the presence of the hemostatic materials are shown in FIG. 11B. The effect of the materials on the platelet contribution to the clot was calculated by subtracting the NAFibTEM A10 value from the NATEM A10 value.

For CTNF0, the sample with the lowest specific surface area and aspect ratio, the CT in the NATEM assay was shortened by 4% (±SE 2%) and in NAFibTEM assay was reduced by 8% (±SE 5%). The contribution of plasma in the A10 with addition of CTNF0 were 26% (±SE 10%), while no significant contribution of platelets were observed (FIG. 11B). This implies that the hemostatic activity of macro-scale chitin fibers relies mostly on the presence of plasma components.

In contrast, CTNF5, the sample with the highest aspect ratio (about 155) and a high specific surface area, shortened CT in the presence and absence of platelet activity, by 70% (±SE 2%) and 74% (±SE 2%), respectively. Additionally, CTNF5 increased the contribution of platelets and plasma to the A10, by 36% (±SE 8%) and 23% (±SE 2%), respectively (FIG. 11B). This infers that the nanoscale features of chitin nanofibers (high specific surface area and aspect ratio) induced the activation of plasma coagulation resulting in more fibrin formation as well as the activation of platelets. As demonstrated in Table 8, the measured plasma and platelet contributions were increased in CTNF5 comparing to CellNF90.

CTNF6, the sample with the highest specific surface area but a low aspect ratio, induced the change in CT by a magnitude similar to that of CTNF5, in the presence and absence of platelet activity. However, the plasma contribution was enhanced by 50% (±SE 4%) and platelets contribution reduced by 15% (±SE 4%) (FIG. 11B).

Celox®, the commercial chitosan-based hemostatic agent made of chitosan powder-coated gauze, was also compared with chitin nanofibers. The NATEM and NAFibTEM studies on the chitosan powder suspension collected from Celox® showed minimal change in clotting profile as well as the contributions of platelets and plasma (FIG. 11).

As chitin nanofibers synthesized by 5 hours of milling is considered to be the optimum sample, their freeze-dried (sponge) form was also studied. The pro-coagulant properties of dry CTNF5 sponge (1.5 mg) were compared with equivalent weights of Celox® using NATEM. The CTNF5 in dry form shortened the clotting time by 63% (±SE 11%) and increased the clot firmness by 6% (±SE 1%) which is less efficient comparing to the suspension form and not significantly better than Celox® (FIG. 11C).

The obtained clots were studied for their microstructure, using SEM (FIG. 11D). In the clot with CTNF5, a mesh-like structure was formed wherein the CTNF5 intermingled with blood components and fibrin fibers, supporting the enhanced plasma coagulation (FIG. 11D). On the other hand, Celox®, made of chitosan powder with positively-charged surface, behaves as a physical barrier and binds red blood cells and platelets, with a distinct absence of fibrin formation.

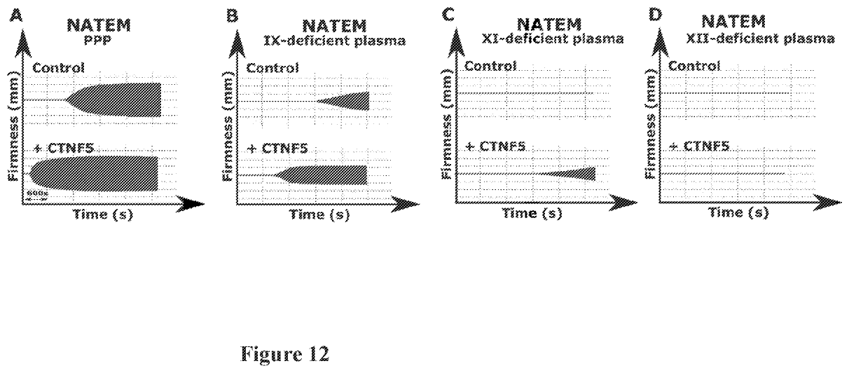

The hemostatic performance of chitin nanofibers in the absence of contact activation plasma factors of IX, XI, and XII were studied using thromboelastometry. The NATEM studies were performed on platelet-poor plasma (PPP) and on plasma deficient of Factor-IX, XI or XII, in the presence and absence of CTNF5 (FIG. 12A-D), and the results were compared with that of CellNF90 (FIGS. 4B and 4C). In PPP without presence of platelets with addition of CTNF5 and CellNF90 in NATEM, a fast clotting time (−79%) and (−71%) was obtained, indicative of enhanced plasma coagulation (FIGS. 12A and 4B). In the absence of Factor-IX or -XI with the addition of CTNF5 in NATEM, the smaller reduction in CT was observed (−52% in case of Factor-IX deficient plasma) (FIGS. 12B and C), while no reduction was observed with addition of CellNF90 (FIG. 4C). The results suggest two possible mechanisms of CTNF5; (i) CTNF5 is a stronger contact activator than CellNF90, or (ii) additional plasma coagulation mechanisms are involved in the pro-coagulant properties of CTNF5.

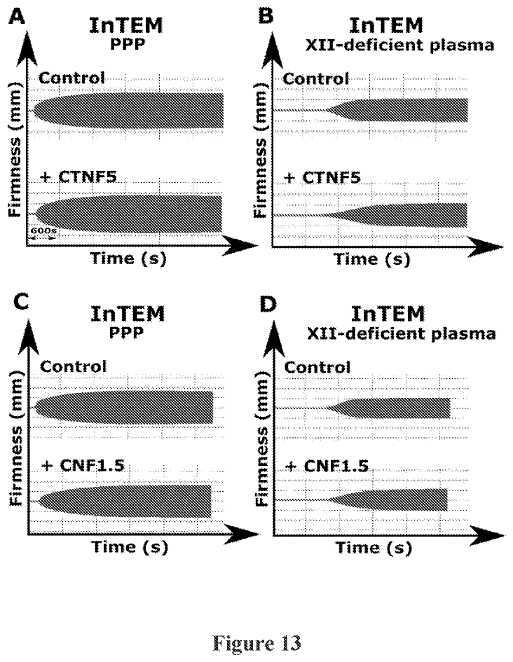

In order to determine which mechanism is more plausible, InTEM studies were performed to trigger contact activation using the InTEM reagent (FIG. 13). In the study, the normal plasma coagulation mechanism was saturated by the higher dosages of contact activation factors, whereby suppressing the contact activation by CTNF5. If CTNF5 has other plasma coagulation mechanisms in addition to contact activation, CTNF5 should further decrease CT even if the contact activation was suppressed. The results show that, with introduction of higher dosages of contact activation factors, the addition of both CTNF5 and CellNF90 (expressed as “CNF1.5” in FIG. 13) has no significant effect on the clotting time, both in PPP and in the plasma deficient of coagulation Factor-XII (FIG. 13). The results imply that no additional plasma coagulation mechanism is involved in the pro-coagulant properties of CTNF5, and that contact activation by CTNF5 is stronger than by CellNF90.

In order to confirm the effect of nanoscale morphology on the pro-coagulant performance of carbohydrate fibers as observed in our previous work on cellulose (as described above), chitin, the second most abundant biomass-based polymer, was studied. Chitin nanofibers with an optimum aspect ratio and specific surface area were produced after 5 hours of ball-milling (FIG. 10). The milling process induced no change in the chemical structure. Compared to the work on cellulose, a longer milling time was required to obtain an optimum morphology in the chitin nanofibers. This is due to the hydrophobic nature of chitin that impede a good dispersion in aqueous media. The use of an acidic milling environment may improve the efficiency to produce chitin nanofibers with shorter milling times and less zirconia contamination. The use of other sources of chitin may also shorten the required ball-milling process, because shrimp-sourced α-chitin used in this work has stronger hydrogen bonding and less reactivity and solubility than other sources of chitin, such as squid and mushroom.

CTNFs with an optimum morphology enhanced hemostasis significantly more than cellulose nanofibers (FIG. 11A), reaching the maximum clot firmness faster. The result was attributable to the difference in the surface charge between chitin (positively charged) and cellulose (negatively charged). The adsorption of red blood cells and platelets on the surface of positively charged chitin is one of the known pro-coagulant mechanisms of macro-scale chitin. The clot formed by chitin nanofibers with a high specific surface area reaches the maximum strength more rapidly than the clot formed by cellulose nanofibres or by macro-scale chitin, with the more rapid adsorption of red blood cells and platelets. However, chitin nanofibers in a dry form do not perform as well as in the suspension form, because the hydrophobic nature of chitin limits its water adsorption capacity and its disintegration in blood (FIG. 11C).

Direct correlations were observed between the contribution of plasma and the specific surface area of chitin nanofibers as well as between the contribution of platelets and the aspect ratio of chitin nanofibers. The results support the findings on cellulose nanofibers with optimum morphology obtained after 1.5 hours of milling, (FIG. 11B).

Nevertheless, the inherent pro-coagulant properties of chitin differ from those of cellulose. Therefore, it is reasonable to expect that the mechanism in which the chitin nanofibers interact with blood is also different from that of cellulose nanofibers. In fact, chitin has a positively-charged surface (zeta potential +26±1 mV at pH 7) while cellulose has a negatively-charged surface (zeta potential −28±1 mV at pH 7). Fibrinogen is known to be negatively charged and to spread out on positively-charged hydrophobic surfaces through electrostatic interactions. Thus, an increase in the specific surface area of chitin nanofibers leads to a greater amount of fibrinogen adsorption and, in turn, more rapid fibrin formation. Platelets are also adsorbed on the hydrophobic and positively-charged surface of chitin. Chitin nanofibers with a higher aspect ratio can provide a mesh like structure to trap an increased number of platelets and, in turn, activate them more efficiently.

In the cellulose studies, cellulose nanofibers with a high specific surface area were shown to enhance plasma coagulation through contact activation. The result was in line with the common belief that contact activation occurs on negatively-charged hydrophilic surfaces. However, in this chitin-based study, contact activation was also observed with positively-charged hydrophobic chitin nanofibers.

### Example 9—Mechanism of Haemostasis by CellNFs

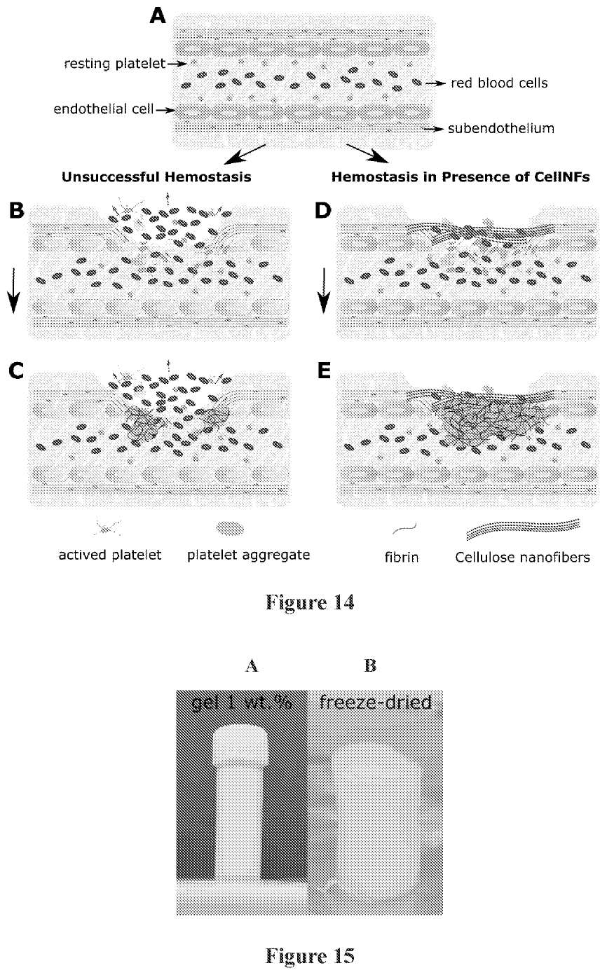

FIG. 14 is a schematic that summarizes the mechanism in which optimized CellNFs contribute to the formation of clot. Unsuccessful haemostasis in the absence of CellNFs is compared to successful hemostasis in the presence of CellNFs.

A healthy blood vessel with the intact endothelial and subendothelial structure, with red blood cells and platelets passing through can be seen in (FIG. 14A). At this stage platelets (with the morphology of small discs) are in the resting condition and are pushed to the vessel edge by the dynamics of blood flow. In the event of trauma, platelets are activated following contact with proteins of the sub-endothelial matrix of the blood vessels, and undergo shape change by growing filopodia. They also release multiple compounds that activate nearby platelets but also interact with plasma coagulation factors (FIG. 14B). As shown in FIG. 14C, platelets spread over the damaged tissue and form the initial platelet plug (platelet activation). Simultaneously, tissue factor on damaged tissue stimulates the plasma coagulation enzymatic cascade ultimately generating thrombin. Thrombin further activates platelets but also cleaves the plasma protein, fibrinogen, enabling it to form long chains known as fibrin. Activated platelets bind to and cross-link the fibrin strands thus forming a strong network in which red blood cells are incorporated. Ultimately, a strong clot is formed at the site of injury preventing further blood loss. However, this complex process is not always successful, especially in the case of a major injury (FIG. 14C).

FIGS. 14D and 14E show the haemostatic process in the presence of our optimised CellNFs which appears to mediate blood coagulation through a combination of three main mechanisms: (i) negatively-charged CellNFs with high specific surface area enhance induction of the intrinsic pathway of plasma coagulation resulting in thrombin generation (ii) CellNFs with high specific surface area adsorb fibrinogen within the plasma thus promoting fibrin strand formation through the actions of thrombin (iii) CellNFs with high aspect ratio form mesh-like physical barriers against, trapping and concentrating, platelets at the location where thrombin and fibrin strands are also focused. The combination of these effects results in the rapid formation of a robust clot in not only healthy blood with/without heparin but also in the blood from thrombocytopenic patients.

### CONCLUSIONS

The synthesis of CellNFs via an environmentally friendly ball-milling method has been demonstrated without altering original characteristics (crystal phase and surface chemical species) of cellulose. The CellNFs showed a significantly improved haemostatic behavior compared to the most common haemostatic agents in both application forms of dry sponge and gel (1 wt. % suspension).

The present systematic studies reveal a direct relationship between enhancement of haemostatic behavior and the physical properties of CellNFs. A higher aspect ratio provides a mesh-like structure similar to natural fibrin, which enables the trapping of platelets, and their partial activation. A larger specific surface area promotes interactions with blood plasma components activating the intrinsic pathway of coagulation with subsequent thrombin formation and fibrin formation thus enhancing haemostasis. The structure-performance relationships enabled the optimum synthesis conditions of CellNFs for superior haemostatic behavior to be identified. In addition, CellNFs provide a physical barrier against blood flow by holding naturally-formed clots firmly and adsorbing the blood fluid. The combination of these mechanisms make CellNFs an excellent haemostatic agent for patients with many different coagulation disorders.

Unlike ORC, CellNFs maintained a neutral pH and did not cause lysis of red blood cells, and did not interfere with the proliferation of endothelial and fibroblast cells. Subcutaneous implantation of the novel ANU hemostat in mice showed histologically a foreign body reaction at 2 weeks which at 4 weeks was significantly resolved with evidence of slow degradation. In contrast to the commercial hemostat, tissue scarring was not evident in case of CellNFs. As such, lower inflammation of the wound sites is expected from CellNFs compared to ORC products.

The present studies also show that chitin, the second most abundant biomass-based carbohydrate polymer, can be ball-milled to produce nanofibers with an optimum morphology for hemostatic applications. After 5 hours of milling, chitin nanofibers with a high aspect ratio and a specific surface area reduced clotting time by 70% (±SE 2%), and increased clot firmness by 12% (±SE 1%). Due to the excellent inherent hemostatic properties of chitin, chitin nanofibers outperformed cellulose nanofibers with a similar morphology. However, the hydrophobic nature of chitin restricts its disintegration in blood and interaction with plasma components and platelets, consequently limits their applications in dry forms.

Systematic in vitro thromboelastometry studies confirmed the structure-performance correlation of chitin nanofibers with blood coagulation system; (i) the high specific surface area of hydrophobic and positively-charged chitin nanofibers enhanced adsorption of blood plasma proteins, such as fibrinogen, and increased fibrin formation through intrinsic pathways despite their positive charge, (ii) the high aspect ratio of chitin nanofibers provided a mesh like structure to entrap platelets and red blood cells.

The cellulose and chitin studies demonstrate that, regardless of the inherent hemostatic properties of materials, the structural characteristics of nanofibers such as aspect ratio and specific surface area, directly influence their pro-coagulant properties. Thus, nanofibers with outstanding hemostatic properties can be potentially produced from many other biomass-based materials.

The anticipated lack of side effects combined with excellent hemostatic effects and the possibility of scalable green production, make CellNFs and CTNFs a new class of promising haemostatic materials. It will be appreciated that, although specific embodiments of the invention have been described herein for the purpose of illustration, various modifications may be made without deviating from the spirit and scope of the invention as defined in the following claims.

1. A haemostatic composition comprising a non-acidic meshed network of fibrous material, wherein the network comprises fibres with a mean diameter (D₅₀) no greater than 1 μm, an aspect ratio (mean fibre length/mean fibre diameter) of at least 100, and wherein said meshed network has a specific surface area of at least 10 m²/g, and a gel point no greater than 3 g/L.

2. The composition of claim 1 wherein said fibrous material is derived from a biopolymer.

3. The composition of claim 2, wherein said biopolymer is a non-oxidised insoluble polysaccharide.

4. The composition of claim 3, wherein said polysaccharide comprises a β-linked backbone.

5. The composition of claim 4, wherein said polysaccharide is non-oxidised high molecular weight cellulose.

6. The composition of claim 5, wherein said material is prepared by mechanical processing of at least partially fractionated biomass rich in high molecular weight cellulose.

7. The composition of claim 6, wherein said mechanical processing comprises ball milling.

8. The composition of claim 1, wherein said fibrous material comprises fibres with a mean diameter less than 100 nm, an aspect ratio of at least 120, and wherein said meshed network has a specific surface area of at least 13 m²/g, and a gel point no greater than 1.5 g/L.

9. The composition of claim 8, wherein said fibrous material comprises fibres with a mean diameter less than 50 nm, an aspect ratio of at least 150, and wherein said meshed network has a specific surface area of at least 15 m²/g, and a gel point no greater than 1.2 g/L.

10. The composition of claim 9, wherein said polysaccharide is non-oxidised high molecular weight cellulose.

11. The composition of claim 10, wherein said material is prepared by mechanical processing of at least partially fractionated biomass rich in high molecular weight cellulose.

12. A haemostatic composition comprising a non-oxidised meshed network of cellulose fibres, said cellulose fibres having a mean diameter (D₅₀) less than 100 nm, an aspect ratio (mean fibre length/mean fibre diameter) of at least 120, and wherein said meshed network has a specific surface area of at least 13 m²/g, and a gel point no greater than 1.5 g/L.

13. The composition of claim 12, wherein said fibrous material comprises fibres with a mean diameter less than 50 nm, an aspect ratio of at least 150, and wherein said meshed network has a specific surface area of at least 15 m²/g, and a gel point no greater than 1.2 g/L.

14. The composition of claim 12, wherein said material is prepared by mechanical processing of at least partially fractionated biomass rich in high molecular weight cellulose.

15. The composition of claim 14, prepared by ball-milling of at least partially fractionated biomass rich in high molecular weight cellulose having a mean degree of polymerization of at least 1000.

16. A method for controlling bleeding from a surface, said method comprising applying to said surface a composition according to claim 1.

17. A method for controlling bleeding from a surface, said method comprising applying to said surface a composition according to claim 11.

18. A method for controlling bleeding from a surface, said method comprising applying to said surface a composition according to claim 12.

19. A haemostatic composition comprising a non-oxidised meshed network of chitin fibres, said chitin fibres having a mean diameter (D₅₀) less than 100 nm, an aspect ratio (mean fibre length mean fibre diameter) of at least 100, and wherein said meshed network has a specific surface area of at least 13m²/g, and a gel point no greater than 1.5 g/L.

20. A method for controlling bleeding from a surface, said method comprising applying to said surface a composition according to claim 19.
